SEQUENCE LISTING (21 sequences)

SEQ ID NO: 1 - protein, 40 aa, Unknown
  Description of Unknown Beta-amyloid peptide
DAEFRHDSGYEVHHQKLVFFAEDVGSNKGAIIGLMVGGVV

SEQ ID NO: 2 - protein, 42 aa, Unknown [sequence omitted]

SEQ ID NO: 3 - protein, 37 aa, Homo sapiens [sequence omitted]

SEQ ID NO: 4 - protein, 21 aa, Homo sapiens
KTNMKHMAGAAAAGAVVGGLG

SEQ ID NO: 5 - protein, 44 aa, Artificial Sequence
  Description of Artificial Sequence Synthetic polypeptide
KKQQQQQQQQQQQQQQQQQQQQQQQQQQQQQQQQQQQQQQQQKK

SEQ ID NO: 6 - protein, 147 aa, Homo sapiens
MKALIVLGLVLLSVTVQGKVFERCELARTLKRLGMDGYRGSLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 7 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 8 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 9 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 10 - protein, 217 aa, Artificial Sequence
  Description of Artificial Sequence Synthetic polypeptide
AQAAELVMTQTPASVSAAVGGTVTINCQSSESVYNSRLSWFQQKPGQPPKLLIYFASTLASGVSSRFSGSGSGTEFTLTISGVQCDDAATYYCAGHFSNSVYTFGGGTEVVVTGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 11 - protein, 226 aa, Artificial Sequence
  Description of Artificial Sequence Synthetic polypeptide
AAQPAMAQSVEESGGRLVTPGTPLTLTCTVSGFSLSAYEVSWVRQAPGKGLEWIGIIYANGNTVYASWAKGRFTISKTSTKVDLRIPSPTTEDTATYFCARDIYTTTTNLWGPGTLVTVSSGQPKAPSVFPLAPCCGDTHSSTVTLGCLVKGYLPEPVTVTWNSGTLTNGVRTFPSVRQSSGLYSLSSVVSVTSSSQPVTCNVAHPATNTKVDKTVAPSTCSKTSC

SEQ ID NO: 12 - protein, 220 aa, Artificial Sequence
  Description of Artificial Sequence Synthetic polypeptide
AQAAELVMTQTPSSVSEPVGGTVTIKCQASQSIDSYLSWYQQKPGQPPKLLIYSASTLASGVPSRFKGSGSGTQFTLTISDLECADAATYYCQSNYWSTSYSYAVPFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 13 - protein, 251 aa, Artificial Sequence
  Description of Artificial Sequence Synthetic polypeptide
AAQPAMAQLVEESGGRLVTPGTPLTLTCTVSGFSLSSYAMSWVRQAPGKGLEWIGVISSSGNTYYASWAKGRFTISKTSTTVDLKMTSLTTEDTATYFCARGVVSGRIDTGLTLWGPGTLVTVSSVSLSSPTLYPLVSCEGALTDGNLVAMGCLARDFLPSSITFSWSFKNNSEISSRTVRTFPVVKRGDKYMATSQVLVPSKDVLQGTEEYLVCKVQHDNSNRDLRVSFPGEPRGLPRDGEVDSELPTSV

SEQ ID NO: 14 - protein, 220 aa, Artificial Sequence; identical to SEQ ID NO: 12
  Description of Artificial Sequence Synthetic polypeptide

SEQ ID NO: 15 - protein, 217 aa, Artificial Sequence
  Description of Artificial Sequence Synthetic polypeptide
AQAAELDMTQTPASVSEPVGGTVTIKCQASQSISSYLAWYQQKPGQRPRLLIYETSTLASGVPSRFKGSGSGTEFTLTISDLECADAATYYCQSTYENPTYVSFGGGTEVGVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 16 - protein, 227 aa, Artificial Sequence
  Description of Artificial Sequence Synthetic polypeptide
AAQPAMAQSVKESGGRLVTPGTPLTLACTVSGFSLNTYSMFWVRQAPGKGLQWIGIISNFGVIYYATWAKGRFTISKTSTTVDLKITSPTTEDTATYFCVRKYGSEWGGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKTSC

SEQ ID NO: 17 - protein, 218 aa, Artificial Sequence
  Description of Artificial Sequence Synthetic polypeptide
AQAAELVMTQTPSSVSAAVGGTVTISCQSSESVYNNNYLSWYQQKPGQPPKRLIDSASTLDSGVPSRFKGSGSGAQFTLTISDLECDDAATYYCAGAYVNWMRIFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 18 - protein, 222 aa, Artificial Sequence
  Description of Artificial Sequence Synthetic polypeptide
MAQSVEESGGRLVTPGTPLTLTCTASGFSLINYYMNWVRQAPGKGLEWIGLITGWADTYYANSAKGRFTISKTSSTTVDLKITSPTTDDTATYFCVRGGHTNIISLWGPGTLVTVSSGQPKAPSVFPLPPCCGDTPSSTVTLGCLVKGYLPEPVTVTWNSGTLTNGVRIFPSVRQSSGLYSLSSVVSVTSSSQPVTCNVAHPATNTKVDKTVAPSTCSKTSC

SEQ ID NO: 19 - protein, 6 aa, Artificial Sequence
  Description of Artificial Sequence Synthetic 6xHis tag
HHHHHH

SEQ ID NO: 20 - protein, 42 aa, Artificial Sequence
  Description of Artificial Sequence Synthetic polypeptide
KKQQQQQQQQQQQQQQQQQQQQQQQQQQQQQQQQQQQQQQQQ

SEQ ID NO: 21 - protein, 36 aa, Artificial Sequence
  Description of Artificial Sequence Synthetic polypeptide
QQQQQQQQQQQQQQQQQQQQQQQQQQQQQQQQQQQQ

(19) United States
(12) Patent Application Publication
(10) Pub. No.: US 2011/0200609 A1
(43) Pub. Date: Aug. 18, 2011

(54) MONOCLONAL ANTIBODIES SPECIFIC FOR PATHOLOGICAL AMYLOID AGGREGATES COMMON TO AMYLOIDS FORMED FROM PROTEINS OF DIFFERING SEQUENCE

(75) Inventors: Charles G. Glabe, Irvine, CA (US); Rakez Kayed, Irvine, CA (US)

(73) Assignee: THE REGENTS OF THE UNIVERSITY OF CALIFORNIA, Oakland, CA (US)

(21) Appl. No.: 13/055,908

(22) PCT Filed: Jul. 27, 2009
(86) PCT No.: PCT/US2009/051880

§ 371 Date: Apr. 18, 2011

Related U.S. Application Data
(63) Continuation-in-part of application No. 10/572,001, filed on Dec. 21, 2006, now abandoned.
Application No. 10/572,001 is a continuation-in-part of application No. 10/527,678, filed on May 9, 2006.
(60) Provisional application No. 61/083,853, filed on Jul. 25, 2008.
(60) Provisional application No. 60/502,326, filed on Sep. 12, 2003.
(60) Provisional application No. 60/410,069, filed on Sep. 12, 2002.

(51) Int. Cl. A61K 39/395; G01N 33/566; C07K 16/18; A61P 25/28; A61P 29/00; A61P 25/00

(52) U.S. Cl. 424/152.1; 436/501; 530/388.2; 435/7.1

(57) ABSTRACT

Monoclonal antibody compositions, methods of production and use. The monoclonal antibodies are specific to conformational epitope(s) of a prefibrillar aggregate(s) which contribute to amyloid fibril formation in human or animal subjects who suffer from amyloid diseases (e.g., Alzheimer's Disease) and the hybridomas and monoclonal antibodies produced therefrom. The monoclonal antibodies are useable for immunization of human or animal subjects against Alzheimer's Disease or other amyloid diseases and/or for the diagnosis or detection of Alzheimer's Disease or other amyloid diseases. The monoclonal antibodies may be administered concomitantly or in combination with anti-inflammatory agents, such as gold or gold containing compounds, to decrease neural inflammation associated with amyloid diseases (e.g., Alzheimer's Disease).

### RELATED APPLICATIONS

This application claims priority to U.S. Provisional Patent Application No. 61/083,853 filed Jul. 25, 2008. Additionally, this application is a continuation in part of U.S. patent application Ser. No. 10/572,001 which is a Section 371 national stage of PCT International Patent Application No. PCT/US04/029946 filed Sep. 13, 2004 which claims priority to U.S. Provisional Patent Application No. 60/502,326 filed on Sep. 12, 2003. The entire disclosure of each such earlier-filed application is expressly incorporated herein by reference.

### FIELD OF THE INVENTION

This invention relates generally to the fields of medicine, immunology and protein biochemistry and more particularly to a) methods for the production of monoclonal antibodies specific to conformational epitope(s) of a prefibrillar aggregate(s) which contribute to amyloid fibril formation in human or animal subjects, b) the hybridomas and monoclonal antibodies produced therefrom, c) the use of such monoclonal antibodies in the immunization of human or animal subjects against Alzheimer's Disease or other amyloid diseases and d) the use of such monoclonal antibodies in the diagnosis or detection of Alzheimer's Disease or other amyloid diseases in human or animal subjects.

### BACKGROUND OF THE INVENTION

Many biological functions come about, at least in part, due to the ability of proteins to adopt various sequence-dependent structures. However, certain protein sequences can sometimes form aberrant, misfolded, insoluble aggregates known as amyloid fibrils. These amyloid fibrils are thought to be involved in the pathogenesis of various amyloid diseases of genetic, infectious and/or spontaneous origin, including spongiform encephalopathies, Alzheimer's disease, Parkinson's disease, type II diabetes, Creutzfeldt-Jakob disease, Huntington's disease, possibly macular degeneration, various prion diseases and numerous others. In at least some of these amyloid diseases, amyloid fibrils lead to the development of amyloid plaques.

Amyloid peptides are the principal constituent of amyloid plaques. In the case of Alzheimer's disease, the peptides are termed Aβ or β-amyloid peptide. Aβ peptide is an internal fragment of 39 to 43 amino acids of amyloid precursor protein (APP). Several mutations within the APP protein have been correlated with the presence of AD. See, for example, Goate et al., Nature, (1991) 349, 704 (valine to isoleucine); Chartier Marian et al., Nature (1991)353, 844 (valine to glycine); Murrell et al. Science (1991) 21, 97 (valine to phenylalanine); Mullan et al., Nature Genet. (1992) 1,345 (a double mutation changing lysine 595-methionine596. to asparagine-595-leucine596). Such mutations are thought to cause AD by producing an increased or altered processing of APP to Aβ. In particular, the processing of APP resulting in accumulation of the longer forms of Aβ, for example, Aβ1-42 and Aβ1-43 is thought to be important in the cause of Aβ. Mutations in other genes, such as the presenilin genes PS1 and PS2, are thought to indirectly affect processing of APP resulting in production of the long form of Aβ. See, for example, Hardy, TINS (1997) 20, 11.

It is believed that cytotoxic amyloid-beta peptide aggregates disrupt the integrity of cell membranes and elaborate reactive oxygen intermediates, thereby giving rise to elevations in cytosolic calcium and eventual cell death. Cell surface receptors for amyloid-beta peptide may also activate signal transduction mechanisms.

European Patent Publication EP 526,511 (McMichael) and PCT International Patent Publication WO/9927944 (Schenk) have described the administration of Aβ to patients for the treatment or prevention of Alzheimer's.

However, although active immunization of Aβ to transgenic mice produces apparent benefits, the extension of this approach to AD patients has resulted in undesirable inflammation of the central nervous system in some of the subjects. See Hardy, D. J. Selkoe (2002) Science 297, 353-356. Soluble Aβ includes Aβ monomers as well as aggregations of such monomers referred to as prefibrillar aggregates. These prefibrillar aggregates lead to the development of amyloid fibrils.

Soluble Aβ content of the human brain is better correlated with the severity of AD than is the accumulation of amyloid plaques. See, for example, Y. M. Kuo et al. (1996) J. Biol. Chem. 271, 4077-4081; C. A. McLean et al. (1999) Annals of Neurology 46, 860-6; L. F. Lue et al. (1999) American Journal of Pathology 155, 853-862. In addition, recent reports suggest that the toxicity of A and other amyloidogenic proteins lies not in the soluble monomers or insoluble fibrils that accumulate, but rather in the prefibrillar aggregates. See, for example, Hartley et al. (1999), Journal of Neuroscience 19, 8876-8884; Lambert et al., Proceedings of the National Academy of Sciences of the United States of America (1998) 95, 6448-53; and Bucciantini et al., Nature (2002) 416, 507-511; and Hartley et al. Nature (2002) 418, 291. Taken together, these results indicate that the prefibrillar aggregates may be more pathologically significant than other forms of the amyloid peptides and therefore may be a more desirable target in the prevention or curing of amyloid diseases such as AD.

PCT International Patent Application PCT/US2003/028829 (WO 2004/024090) entitled MONOCLONAL ANTIBODIES AND CORRESPONDING ANTIBODIES SPECIFIC FOR HIGH MOLECULAR WEIGHT AGGREGATION INTERMEDIATES COMMON TO AMYLOIDS FORMED FROM PROTEINS OF DIFFERING SEQUENCE (Kayed and Glabe) describes compositions of matter comprising one or more conformational epitopes found on amyloid peptide aggregates, antibodies to such epitopes and methods for making and using the compositions, epitopes and/or antibodies. The compositions described in PCT/US2003/028829 include synthetic or isolated compositions that contain or consist of certain conformational epitopes found on peptide aggregates (e.g., toxic peptide aggregates) present in human or veterinary patients who suffer from, or who are likely to develop, amyloid diseases (e.g., Alzheimer's Disease). The invention described in PCT/US2003/028829 also includes methods for using such compositions in the detection, treatment and prevention of diseases in humans or animals and/or in the testing and identification of potential therapies (e.g., drug screening) using such antibodies. The entirety of PCT International Patent Application PCT/US2003/028829 is expressly incorporated herein by reference.

Monoclonal antibodies are homogeneous preparations of immunoglobulin proteins that specifically recognize and bind to regions, or epitopes, of their corresponding antigens. In some cases, monoclonal antibodies can bind to and inhibit the activity of endogenous chemical entities that are toxic or deleterious. In view of this, there is a need for the development of new monoclonal antibodies that bind to and inhibit toxic forms of amyloid (e.g., cytotoxic amyloid-beta peptide aggregates or protofibrils) with high specificity, thereby providing for diagnosis and treatment of amyloid diseases.

U.S. patent application Publication 007/0218499 (Lambert et al.) entitled MONOCLONAL ANTIBODIES THAT TARGET PATHOLOGICAL ASSEMBLIES OF AMYLOID β (Aβ) describes monoclonal antibodies that purportedly bind with high specificity to soluble oligomers of amyloid β peptide. (Aβ) and methods of employing those antibodies. The antibodies are described to be able to distinguish between Alzheimer's Disease (AD) and control human brain extracts. The antibodies identify endogenous Aβ oligomers in AD brain slices and also bind to Aβ oligomers on cultured hippocampal cells. The antibodies neutralize endogenous Aβ oligomers and Aβ oligomers produced in solution.

As shown in the following diagram, there exist alternative pathways and assembly states of Aβ and other amyloids. Amyloid formation begins with amyloidogenic or partially folded monomer (dark blue) which has the ability to aggregate. It can form fibrils by forming soluble fibril nuclei (light blue) which can grow by addition of monomers to form insoluble fibrils (light blue). Fibrillar oligomers and fibrils are recognized by a fibril specific polyclonal serum, “OC”. Alternatively, monomers can aggregate to form prefibrillar oligomers that are immunologically distinct from fibrils and monomer. The oligomers co-aggregate to form curvilinear protofibrils and may ultimately undergo a conformation change to form fibrils. Prefibrillar oligomers and protofibrils are specifically recognized by A11. Prefibrllar oligomers also appear to be building blocks for annular protofibrils that appear to be pore like structures. Annular protofibrils are preferentially recognized by “officer” polyclonal serum.

The hallmark lesions of AD include amyloid deposits, neurofibrillary tangles, and dystrophic neurites. Several types of amyloid deposits are found including diffuse amyloid deposits, “cored”, “neuritic” and “compact or burned out” senile plaques and cerebrovascular amyloid deposits. Neurofibrillary tangles, comprised of the misfolded microtubule-associated protein tau, are frequently found frequently in association with dystrophic neurites. The linkage of familial AD mutations to the increased production of more highly aggregation prone Aβ42 support a causal role of Aβ aggregation in disease, but the precise relationships between aggregation state and disease remain to be established. There is conflicting evidence for the role of large extracellular aggregates or plaques in pathogenesis. It has been reported that the extent of amyloid plaque accumulation does not correlate well with Alzheimer's disease pathogenesis and that a significant numbers of non-demented individuals have significant amounts of amyloid plaques. Other studies have reported a positive correlation between the extent of amyloid plaque deposition, cognitive dysfunction and plaque associated disruption of neuritic morphology and toxicity. In some transgenic animals and cell culture models, pathological changes are frequently observed prior to the onset of amyloid plaque accumulation. It has also been reported that soluble Aβ correlates better with dementia than insoluble, fibrillar deposits, suggesting that oligomeric forms of Aβ may represent the primary toxic species in AD.

Aggregates ranging from dimers up to particles of a million Da or greater have been reported in vitro. Electron microscopy and atomic force microscopy have identified spherical particles of approximately 3-10 nm that appear at early times of incubation and disappear as mature fibrils appear. These spherical oligomers appear to represent intermediates in the pathway of fibril formation because they are transiently observed at intermediate times of incubation during fibril formation. Soluble Aβ oligomers have been referred to as amorphous aggregates, micelles, protofibrils, prefibrillar aggregates, ADDLs, Aβ*56, globulomers, and “tAβ” (toxic soluble Aβ). At longer aggregation times, curvilinear fibers form that have a beaded appearance form. These structures have also been called “protofibrils” because they appear to be formed by the coalescence of the spherical subunits. Pore-like structures, known as “annular protofibrils” have been observed in solutions of oligomers. This same spectrum of aggregation intermediates and morphologies have been observed for many types of amyloids, such as alpha synuclein, islet amyloid and non-disease associated “neoamyloids”. Finally, these protofibrillar structures appear to either anneal or undergo a conformational change to form mature 6-10 nm cross beta fibrils that have either a smooth or twisted morphology. Once the amyloid fibril lattice has been established, it can grow by the addition of monomer onto the ends of the fibrils. Although these various aggregates have been observed for many different types of amyloids, their structures, the relationships of the aggregation intermediates, their roles in fibril growth and their contributions to disease pathogenesis are not clear. Applicants have endeavored to clarify the number of distinct aggregations states, their structures and their toxicity or pathogenic activities and this is the focus of this application.

There is evidence indicating that soluble Aβ oligomers are more toxic to cells than mature fibrils. Although there is some general agreement that soluble Aβ assembly states are toxic, the size, conformation and pathological significance of the soluble assembly states are topics of current debate. Aβ oligomers secreted into the culture medium run as predominantly trimers and tetramers on SDS gels, while a species with an apparent mass of 56 KDa (Aβ*56) is highly correlated with cognitive deficits in Tg2576. Whether these species represent unique structures with distinct toxicities or whether they are part of a broad size spectrum of related toxic structures is not yet clear. Moreover, since SDS used in gel electrophoresis dissociates proteins, the size of these assembly states under native conditions is also a significant issue that remains to be established. Increasing evidence indicates that soluble amyloid oligomers are generally toxic for a wide variety of disease related amyloids. Soluble oligomers are generically toxic because oligomers formed by proteins that are not disease related are equally toxic as disease-related oligomers. The idea that soluble Aβ oligomers are the primary toxic species is an attractive explanation that can reconcile the seemingly inconsistent genetic evidence that amyloids are causally related to disease, while some studies have indicated that the large fibrillar deposits are not. However the evidence in support of this hypothesis is largely correlative and derived from in vitro studies.

It is difficult to directly assess the role of assembly states in disease pathogenesis in complex mixtures because the oligomers only differ from the native protein, soluble monomer and mature fibrils in conformation or aggregation state. To study these differences in vivo, there must be some way to distinguish these assembly states. Conformation dependent monoclonal antibodies, as described in this provisional patent application, have the ability to distinguish native and pathological states.

Therefore, antibodies that distinguish between the different assembly states are very useful for investigating their role in pathogenesis. We produced a conformation-dependent antibody that is specific for soluble prefibrillar oligomers and does not recognize natively folded proteins, monomer, nor fibrils. Surprisingly, this antibody also recognizes soluble oligomers from a wide variety of amyloid forming peptides and proteins equally well. This suggests that the antibody recognizes an epitope that is common to amyloid oligomers of different sequences and is independent of the amino acid side chains. One of the problems intrinsic to studies of in vitro toxicity of Aβ assembly states is that while you may have a good idea of the size and structure of an aggregate when you add it to cells, it is difficult to be sure that it has not aggregated further or changed conformation during the time required to read out measurements of toxicity. The anti-oligomer antibody blocks the toxicity soluble prefibrillar oligomers in vitro, so we will use this blocking ability to confirm the conformation of the active aggregation state as it is presented to cells. The availability of pure and homogeneous populations of different assembly states of Aβ and novel monoclonal antibodies that specifically recognize these assembly states affords us a unique opportunity to identify distinct aggregation states and clarify their roles in Aβ fibril assembly and cellular toxicity.

### SUMMARY OF THE INVENTION

The present invention provides compositions comprising isolated monoclonal antibodies which bind to one or more conformational epitope(s) of prefibrillar aggregate(s) that contribute to amyloid fibril formation in the brains of humans or animals (e.g., toxic species of prefibrillar aggregate(s)). The monoclonal antibodies may be administered, in therapeutic amounts, to human or animal subjects to reduce the toxicity of the prefibrillar aggregate, thereby preventing or limiting the formation of amyloid deposits and the associated occurrence or progression of a disease or disorder in which amyloid deposits form within the brain or nervous tissue. Examples of such amyloid diseases include, but are not necessarily limited to, Alzheimer's Disease, early onset Alzheimer's Disease associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis. The monoclonal antibodies of the present invention are identified as follows: M118, M121, M201, M204, M205, M206 These clones were prepared by immunizing rabbits with a conformationally-constrained antigen consisting of amyloid Aβ covalently coupled to colloidal gold via a thioester linkage.

In accordance with the invention, the prefibrillar aggregate may have a molecular weight in a range of about 9 kDa to about 100,000,000 kDa. Also, the prefibrillar aggregate may comprise any suitable number of monomers. For example, in some specific embodiments the prefibrillar aggregate may comprise five monomers and in other embodiments, the prefibrillar aggregate may comprise eight monomers.

Further in accordance with the present invention, there are provided rabbit monoclonal antibodies and methods as described herein.

Still further in accordance with the invention, the amyloid peptide monomers and/or amyloid fibrils may be substantially free of the conformational epitope to which the monoclonal antibodies M118, M201, M204, M205 and M206 binds. Similarly, amyloid peptide monomers and prefibrillar oligomers may be substantially free of the conformational epitope to which monoclonal antibody M121 binds because this antibody is specific for fibrils and fibrillar oligomers or fibril nuclei.

Still further aspects and objects of the present invention may be understood from the detailed description and examples set forth here below.

### BRIEF DESCRIPTION OF THE DRAWINGS

FIG. 1 shows dot blot analysis of specificity for a number of rabbit monoclonal antibodies of the present invention.

FIG. 2A shows dot blot analysis of Aβ40 and Aβ42 incubated under three different conditions.

FIG. 2B shows dot blot analysis of four different samples of prefibrillar oligomers prepared at pH 2.5 and stained with A11, M204 and M205.

FIG. 2C shows a Western blot of Aβ42 prefibrillar oligomers stained with A11 and M204.

FIG. 3A shows a dot blot analysis of Aβ40 prefibrillar oligomers (1), soluble monomer (2) and fibrils (3) probed with anti-oligomer (top) or stripped and reprobed with 6E10 (bottom).

FIG. 3B is a graph showing results of an ELISA assay wherein increasing amounts of Aβ40 prefibrillar oligomers (open circles) monomer (filled triangles) and fibrils (filled squares) were applied to the wells and developed with anti-oligomer antibody.

FIG. 4A shows an ELISA assay of different types of fibrils and soluble Aβ monomer and prefibrillar oligomers.

FIG. 4B shows a dot blot of Aβ42 fibrils and prefibrillar oligomers stained with anti-fibril (OC) or anti-oligomer (A11).

FIG. 4C shows a Western blot of fibrillar (F) and prefibrillar oligomer (O) Aβ42 samples.

FIG. 5A shows negative stained electron micrograph of relatively homogeneous populations of Aβ42 annular protofibrils prepared from solutions of prefibrillar oligomers by treatment with 5% hexane in water.

FIG. 5B shows the specificity of anti-annular protofibrils antisera. Homogeneous samples of Aβ42 annular protofibrils, spherical oligomers, fibrils and monomer (soluble) were plated in the wells of an ELISA plate and reacted with anti-annular protofibrils antibody.

FIG. 5C shows that the anti-annular protofibrils antibody reacts efficiently with annular protofibrils from alpha synuclein and IAPP (amylin).

FIG. 5D shows immunoprecipitation of soluble AD brain extract with anti-oligomer antibody.

FIG. 5E shows immunoprecipitation of non-demented control brain extract with anti-annular protofibril antibody.

FIG. 6A shows the structure of monoclonal M204 Fab as determined by x-ray crystallography in a view (View 1) wherein the antibody fragment with the antigen combining site faces forward toward the viewer.

FIG. 6B shows the structure of monoclonal M204 Fab as determined by x-ray crystallography in a view (View 2) wherein the antibody fragment with the antigen combining site faces toward the viewer's right.

FIG. 7A is a bar graph comparing the initial latency to cross the platform location in a Morris Wayer Maize test of animals treated with Control, anti-oligomer A11, 204 antibody and 205 antibody.

FIG. 7B is a bar graph comparing the time spent during training in the quadrant opposite to the one containing the platform in a Morris Wayer Maize test of mice treated with Control, A11, 204 and 205.

FIG. 7C is a bar graph comparing the numbers of platform location crosses in a Morris Wayer Maize test of mice treated with Control, A11, 204 and 205.

FIG. 8 is a bar graph showing Recognition Index (RI) determined by the Object Recognition test of mice treated with Control, A11, 204 and 205.

FIG. 9A is a bar graph of comparing the mean latency (seconds) in an electroshock passive avoidance test of mice treated with Control, A11, 204 and 205.

FIG. 10 is a bar graph showing Aβ plaque load determined by 6E10 antibody uptake in brain tissue of mice treated with Control, A11, 204 and 205.

### DETAILED DESCRIPTION AND EXAMPLES

### Definitions

As used in this provisional patent application, the following terms shall have the following meanings:

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

The term “Aβ” or “Aβ peptide” refers to peptides which comprise low molecular weight soluble oligomers, prefibrillar aggregates, fibrils and amyloid deposits each associated with AD. Amyloid Aβ peptides include, without limitation, Aβ 39, Aβ 40, Aβ 41 Aβ 42 and Aβ 43 which are 39, 40, 41, 42 and 43 amino acid amino acids in length, respectively.

An “amyloid peptide” is a peptide that is present in amyloid forms including amyloid peptide intermediates, low molecular weight soluble oligomers, amyloid fibrils and amyloid plaques.

The term “antibody” is used to include intact antibodies and binding fragments thereof, including but not limited to, for example, full-length antibodies (e.g., an IgG antibody) or only an antigen binding portion (e.g., a Fab, F(ab′)₂ or scFv fragment). Typically, fragments compete with the intact antibody from which they were derived for specific binding to an antigen. Optionally, antibodies or binding fragments thereof, can be chemically conjugated to, or expressed as, fusion proteins with other proteins.

“Anti-oligomer antibody” or “Anti-oligomer” refer to an antibody that binds to amyloid peptide aggregate intermediates but does not bind to or does not specifically bind to amyloid peptide monomers, dimers, trimers or tetramers.

Compositions or methods “comprising” one or more recited elements may include other elements not specifically recited. For example, a composition that comprises an amyloid Aβ peptide may encompass both an isolated amyloid peptide as a component of a larger polypeptide sequence or as part of a composition which includes multiple elements.

The term “epitope” or “antigenic determinant” refers to a site on an antigen to which B and/or T cells respond or a site on a molecule against which an antibody will be produced and/or to which an antibody will bind. For example, an epitope can be recognized by an antibody defining the epitope.

A “linear epitope” is an epitope wherein an amino acid primary sequence comprises the epitope recognized. A linear epitope typically includes at least 3, and more usually, at least 5, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the antibody defining the epitope). Typically a conformational epitope comprises an increased number of amino acids relative to a linear epitope. With regard to recognition of conformational epitopes, the antibody recognizes a 3-dimensional structure of the peptide or protein. For example, when a protein molecule folds to form a three dimensional structure, certain amino acids and/or the polypeptide backbone forming the conformational epitope become juxtaposed enabling the antibody to recognize the epitope. Methods of determining conformation of epitopes include but are not limited to, for example, x-ray crystallography 2-dimensional nuclear magnetic resonance spectroscopy and site-directed spin labeling and electron paramagnetic resonance spectroscopy. See, for example, Epitope Mapping Protocols in Methods in Molecular Biology, Vol. 66, Glenn E. Morris, Ed. (1996), the disclosure of which is incorporated in its entirety herein by reference.

The term “immunological response” or “immune response” relates to the development of a beneficial humoral (antibody mediated) and/or a cellular (mediated by antigen-specific T cells or their secretion products) response directed against an amyloid peptide in a recipient patient. Such a response can be an active response induced by administration of monoclonal antibody or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity.

A “monoclonal antibodyic agent” or “monoclonal antibody” or “antigen” is capable of inducing an immunological response against itself upon administration to a subject, optionally in conjunction with an adjuvant.

“Isolated” means purified, substantially purified or partially purified. Isolated can also mean present in an environment other than a naturally occurring environment. For example, an antibody that is not present in the whole blood serum in which the antibody would ordinarily be found when naturally occurring is an isolated antibody.

“Low molecular weight aggregate”, “low molecular weight amyloid aggregate”, “low molecular weight oligomer” and “low molecular weight soluble oligomer” refer to amyloid peptides present in aggregates of less than four or five peptides. In one specific example, low molecular weight Aβ refers to the low molecular weight soluble oligomers found associated with AD.

The term “patient” includes human and other animal subjects that receive therapeutic, preventative or diagnostic treatment or a human or animal having a disease or being predisposed to a disease.

“Prefibrillar aggregates”, “micellar aggregates”, “high molecular weight aggregation intermediates,” “high molecular weight amyloid peptide aggregates”, “high molecular weight soluble amyloid peptide aggregates” “amyloid peptide aggregates”, “soluble aggregate intermediates”, “amyloid oligomeric intermediates”, “oligomeric intermediates” and “oligomeric aggregates” or simply, “intermediates” refer to aggregations which include more than three individual peptide or protein monomers, for example, more than four peptide or protein monomers. The upper size of prefibrillar aggregates includes aggregations of oligomers which form spherical structures or micelles and stings of micelles which lead to fibril formation.

“Annular protofibrils” are a particular subset of prefibrillar aggregates in which 3 to 10 spherical oligomer subunits are arranged in an annular or circular fashion with a hollow center that appears as a pore in electron or atomic force micrographs.

The molecular weight of a prefibrillar aggregate may be in a range of about 10 kDa to about 100,000,000 KDa, for example, about 10 kDa to about 10,000,000 or 1,000,000 KDa. However, this size range is not intended to be limiting and prefibrillar aggregates are not defined by a molecular weight range.

“Protofibrils” are prefibrillar aggregates which include spherical structures comprising amyloid Aβ peptides that appear to represent strings of the spherical structures forming curvilinear structures.

“Specific binding” between two entities means an affinity of at least 10⁶, 10⁷, 10⁸, 10⁹ M⁻¹, or 10¹⁰ M⁻¹. Affinities greater than 10⁸ M⁻¹ are preferred for specific binding.

The term “substantial identity” means that two peptide sequences, when optimally aligned, such as by the programs GAP or BESTFIT using default gap weights, share at least 65 percent sequence identity, for example, at least 80 percent or 90 percent sequence identity, or at least 95 percent sequence identity or more, for example, 99 percent sequence identity or higher.

Preferably, residue positions in an alignment which are not identical differ by conservative amino acid substitutions, i.e., substitution of an amino acid for another amino acid of the same class or group. Some amino acids may be grouped as follows: Group I (hydrophobic side chains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Non-conservative substitutions may include exchanging a member of one of these classes for a member of another class.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm may then be used to calculate the percent sequence identity for the test sequence (s) relative to the reference sequence, based on the designated program parameters. Optimal alignment of sequences for comparison can be conducted, for example, by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix, see for example, Henikoff & Henikoff, Proc. Natl. Acad. Sci. USA 89,10915 (1989). Conservative substitutions involve substitutions between amino acids in the same class.

A “therapeutic agent” or “therapeutic” is a substance useful for the treatment or prevention of a disease in a patient. Therapeutic agents of the invention are typically substantially pure. This means that an agent is typically at least about 50% w/w (weight/weight) pure, as well as being substantially free from proteins and contaminants which interfere with the efficacy of the therapeutic. The agents may be at least about 80% w/w and, more preferably at least 90% w/w or about 95% w/w in purity. However, using conventional protein purification techniques, homogeneous peptides of 99% w/w or more can be produced.

### Embodiments and Examples

Amyloid diseases are characterized by the accumulation of amyloid plaques or precursors to amyloid plaques in patients or the predisposition to the accumulation of amyloid plaques or precursors to amyloid plaques in patients. One of the primary constituents of amyloid plaques are amyloid peptides. The general conformation of amyloid peptides may vary from disease to disease, but often the peptide has a characteristic-pleated sheet structure. Amyloid peptides include peptides and proteins of about 10 or about 20 amino acids to about 200 amino acids in length. Though this size range is not intended as a limitation and amyloid peptides or proteins having fewer or more amino acids are contemplated in the present invention.

Prefibrillar aggregates are intermediates in the production of insoluble fibrils that accumulate in amyloid plaques of humans or animals having a disease characterized by amyloid deposits, for example, Alzheimer's disease. Prefibrillar aggregates include aggregates which may be as small as four amyloid peptides, as small as five amyloid peptides, as small as six amyloid peptides, as small as seven amyloid peptides or as small as eight amyloid peptides. In one embodiment, prefibrillar aggregates are micellar aggregates or micelles or strings of micelles. Prefibrillar aggregates are effective to form a conformational epitope which is recognized by an antibody of the present invention.

The conformational epitopes found on prefibrillar aggregates are substantially not found in the native precursor proteins for amyloid peptides, for example, amyloid peptide monomers, the amyloid precursor protein, nor in the amyloid fibrils that are defined by their characteristic cross β x-ray fiber diffraction pattern or in amyloid plaques. The prefibrillar aggregates that contain the specific polypeptide structure which results in conformational epitopes that are recognized by antibodies of the present invention have a size range of approximately a dimer, trimer, tetramer, pentamer, a hexamer, a heptamer or an octamer, dodecamer to micellar forms or protofibrils which have a molecular weight in excess of 1,000,000 Daltons. Antibodies of the invention are effective to bind to these epitopes.

Monoclonal antibodies of the present invention are specific for a conformation-dependent epitope associated with amyloid oligomers or protofibrils or in the case of M121, amyloid fibrils.

### Examples of Rabbit Monoclonal Antibodies

The monoclonal antibodies may be prepared by immunizing mice with a conformationally-constrained antigen consisting of amyloid Aβ covalently coupled to colloidal gold via a thioester linkage. Such monoclonal antibodies will provide for diagnostic and therapeutic uses. The antibody is also useful for determining the three dimensional structure of amyloid oligomers bound to the antibody by co-crystallization of the antibody Fab with the antigen and X-ray crystallography.

Supernatants from hybridoma fusions were sent to UCI by Epitomics, Inc. and screened by ELISA by Dr. Rakez Kayed, Monica Siegenthaler and Maya Hatch by dot blot assay. For ELISA assay, 100 ng of soluble oligomeric or fibrillar Aβ42 was suspended in plating buffer and used to coat hyBond ELISA plates for 1 hr to overnight. After coating the wells were blocked with 300 ul 10 BSA in Tris-buffered saline, 0.01% Tween 40 (TBST) at 37 degrees C. for 1 hr. Tissue culture supernatant from the hybridomas was added to the wells at 1:200, 1:500, 1:1000, 1:2000 and 1:5000 and incubated at 37 degrees for 1 hr. The plates were washed 3× with phosphate buffered saline (PBS) and 100 ul of goat anti rabbit-horseradish peroxidase conjugate 1:10,000 dilution was added to each well and incubated for 1 hr. the plates were washed 3 times with PBS and then assayed for HRP activity by adding 100 ul of color diction substrate, TMB. The plates were read at 450 nm. Clones that show high reactivity against oligomers and low reactivity against monomer and fibrils were selected.

Appendix A, which forms a part of this specification, describes in detail the production of certain monoclonal antibodies of the present invention.

Dot Blot Assay:

Monomer, oligomer and fibrillar samples of Aβ42 (100 ng) were applied to a nitrocellulose membrane, dried and blocked with 10% BSA in TBST. Tissue culture supernatant from the hybridomas was added to each strip at 1:200, 1:500, 1:1000, 1:2000 and 1:5000 and incubated at 37 degrees for 1 hr. The strips were washed 3 times with PBS, and incubated at 37 degrees for 1 hr with goat anti mouse-horseradish peroxidase conjugate 1:10,000. The strips were washed 3 times with PBS and the antibody binding visualized by enhanced chemiluminescence (ECL). A typical dot blot is shown in FIG. 1 for clones M118, M121, M201, M204, M205 and M206. Lane 1 is Aβ42 monomer. Lane 2 is A1142 prefibrillar oligomers. Lane 3 is Aβ42 fibrils. Lane 4 is alpha synuclein prefibrillar oligomers, Lane 5 is immunoglobulin light chain prefibrillar oligomers, Lane 6 is prion 106-126 prefibrillar oligomers, Lane 7 is KK(Q40)KK prefibrillar oligomers, Lane 8 is calcitonin prefibrillar oligomers.

Each of the following amyloid peptides have been shown to form amyloid peptide aggregates which produce a conformational epitope recognized by the antibodies of the present invention, for example, antibodies produced against Aβ peptide oligomeric intermediates. Some of these peptides are present in amyloid deposits of humans or animals having a disease characterized by the amyloid deposits. The present invention is not limited to the listed peptide or protein sequences or the specific diseases associated with some of the sequences. The present invention contemplates antibodies as described herein binding to other amyloid peptide aggregates or all other amyloid peptide aggregates. In particular, the present invention contemplates and includes the application of methods and compositions of the present invention to other peptide or protein sequences which form amyloid precursor aggregates associated with other diseases.

Aβ40
(SEQ ID NO 1)
DAEFRHDSGYEVHHQKLVFF AEDVGSNKGA IIGLMVGGVV
Aβ42
(SEQ ID NO 2)
DAEFRHDSGY EVHHQKLVFF AEDVGSNKGA IIGLMVGGVV IA
Human IAPP
(SEQ ID NO 3)
KCNTATCATQ RLANFLVHSS NNFGAILSST NVGSNTY
Human Prion 106-126
(SEQ ID NO 4)
KTNMKHMAGA AAAGAVVGGL G

Stefani and coworkers (Bucciantini et al (2002) Nature 416, 507-511) have recently reported that amyloid peptide aggregates formed from non-disease-related proteins are inherently cytoxic, suggesting that they may have a structure in common with disease related amyloid peptides. Non-disease related amyloid peptide aggregates comprising the following non-disease related amyloid peptides are also shown to bind to the antibodies of the present invention.

Poly glutamine synthetic peptide KK(Q40)KK
(SEQ ID NO 5)
KKQQQQQQQQ QQQQQQQQQQ QQQQQQQQQQ QQQQQQQQQQ QQKK
Human Lysozyme
(SEQ ID NO 6)
MKALIVLGLV LLSVTVQGKV FERCELARTL KRLGMDGYRG
SLANWMCLA KWESGYNTRA TNYNAGDRST DYGIFQINSR
YWCNDGKTPG AVNACHLSCS ALLQDNIADA VACAKRVVRD
PQGIRAWVAW RNRCQNRDVR QYVQGCGV
Human Insulin
(SEQ ID NO 7)
MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN
Human Transthyretin
(SEQ ID NO 8)
MASHRLLLLC LAGLVFVSEA GPTGTGESKC PLMVKVLDAV
RGSPAINVAV HVFRKAADDT WEPFASGKTS ESGELHGLTT
EEEFVEGIYK VEIDTKSYWK ALGISPFHEH AEVVFTANDS
GPRRYTIAAL LSPYSYSTTA VVTNPKE
Human Alpha Synuclein
(SEQ ID NO 9)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

In addition, oligomeric intermediates formed from variants and fragments of wild type Aβ42, Aβ40 including, without limitation Aβ42 (A21G) Flemish mutation), Aβ42 (E22Q) Dutch mutation, Aβ42 (E22G) Arctic mutation, Aβ42 (D23N) Iowa mutation, Aβ40 (A21G) Flemish mutation), Aβ40 (E22Q) Dutch mutation, Aβ40 (E22G) Arctic mutation, M40 (D23N) Iowa mutation, Aβ40 (E22Q & D23N) Dutch & Iowa mutations, Aβ 3-42 (pGlu 3), Aβ 3-40 (pGlu 3), Aβ8-42, Aβ17-42, Aβ1-16, Aβ3-11, Aβ25-35, Aβ4-16 (3 analogues, Cys¹⁶ Aβ4-16, Ala⁴ Aβ4-16, and Ala¹⁰ Aβ4-16), His6 Aβ40C40 (6 histidines appended to the amino terminus of AβC40) are recognized by the antibodies of the present invention. Other oligomeric intermediates recognized by antibodies of the invention include, without limitation, oligomeric intermediates formed from IAPP(C2A and C7A) where alanine is substituted for the naturally occurring cysteine in IAPP, Polyglutamine KKQ40KK or poly glutamine where the number of Q residues is greater than 32, Calcitonin, TTR and its mutants TTR Pro⁵⁵_{, TTR Phe}⁷⁸, vitronectin, poly Lysine, poly arginine, serum amyloid A, cystatin C, IgG kappa light chain, oligomeric intermediates produced from other amyloid peptides disclosed herein and amyloid intermediates associated with amyloid diseases disclosed herein.

### Examples of Rabbit Monoclonal Antibodies

FIG. 1 shows dot blot analysis of specificity for a number of rabbit monoclonal antibodies of the present invention. Nitrocellulose strips containing 1 ug dots of Aβ42 monomer, prefibrillar oligomers or fibrils and 5 additional types of prefibrillar oligomers were stained with polyclonal A11 and 6 distinct types of rabbit monoclonal antibodies. A11 stains all types of prefibrillar oligomers as previously reported, however, the rabbit monoclonal antibodies display a more restricted specificity than A11 and have distinct preferences for different types of oligomers. For example, M204 recognizes all types of oligomers, but it reacts only weakly with light chain oligomers. M201 only recognizes Aβ oligomers. M118, M205 and M206 have intermediate degrees of specificity and display distinct preferences. M121 is specific for Aβ fibrils and does not recognize Aβ monomer or prefibrillar oligomers. M201 and M121 are IgMs, while the other monoclonals are IgG.

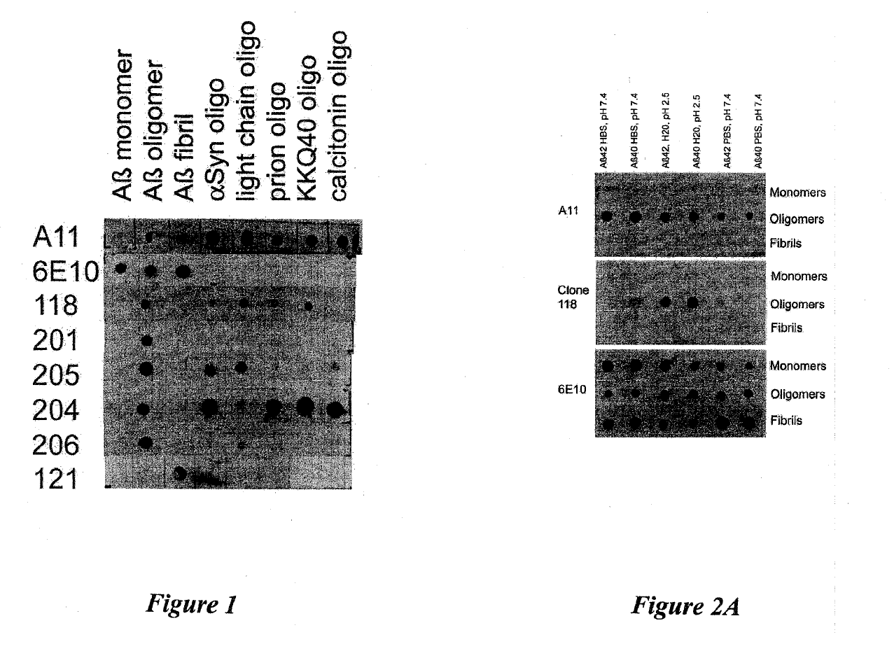

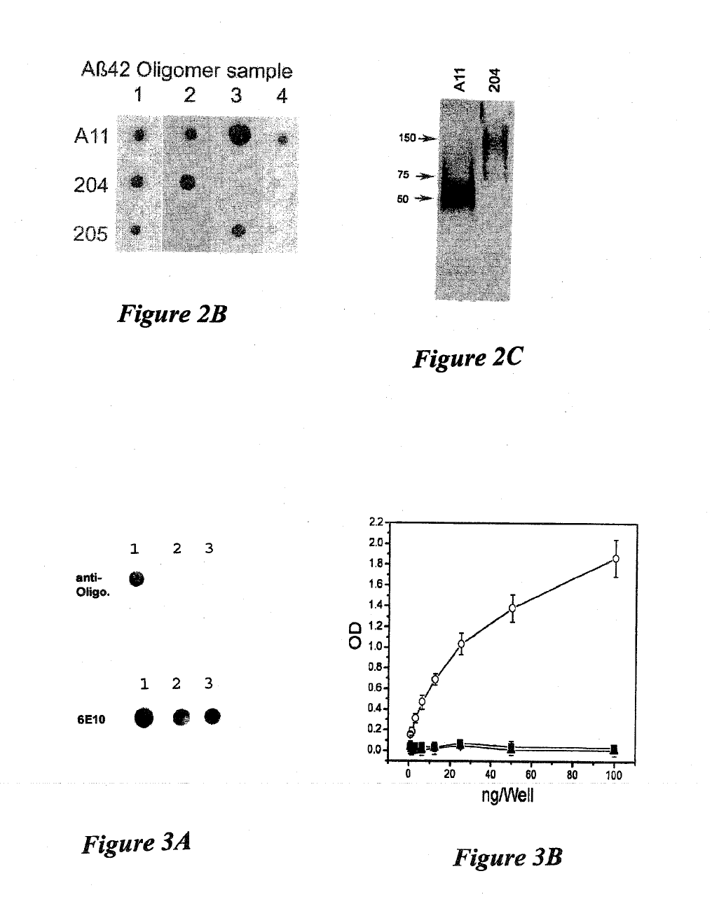

As may be appreciated from FIG. 2, antibodies of the present invention distinguish different subclasses of Aβ prefibrillar oligomers. In FIG. 2A, Aβ40 and Aβ42 were incubated under three different conditions (HBS, hepes buffered saline pH7.4; water pH 2.5; or PBS pH 7.4). Monomer samples were spotted at time zero, while prefibrillar oligomers and fibrils were spotted at 72 and 240 hours of incubation respectively. All of the oligomer samples stain with A11, but M118 only reacts with samples prepared at pH 2.5. In FIG. 2B, four different samples of prefibrillar oligomers were prepared in water pH 2.5 and stained with A11, M204 and M205. All samples stain with A11. Sample 1 stains with both 204 and 205 antibodies while sample 2 stains with only M204, sample 3 stains with M205 and sample 4 stains with neither monoclonal. FIG. 2C shows a Western blot of Aβ42 prefibrillar oligomers stained with A11 and M204. Although the sample stains with both antibodies, the oligomer bands stained by M204 are larger than those stained by A11, indicating that the distinct sizes are also immunologically distinct.

The specificity of anti-prefibrillar oligomer (A11) antibody is demonstrated by FIGS. 3A and 3B. In the dot blot of FIG. 3A, 1 ug each of Aβ40 prefibrillar oligomers (1), soluble monomer (2) and fibrils (3) was spotted on a nitrocellulose membrane and probed with anti-oligomer (top panel) or stripped and reprobed with 6E10 (bottom panel). In FIG. 3B is a graph showing results of an ELISA assay wherein increasing amounts of Aβ40 prefibrillar oligomers (open circles) monomer (filled triangles) and fibrils (filled squares) were applied to the wells and developed with anti-oligomer antibody. The same prefibrillar oligomer specificity was obtained for amylin (IAPP), alpha synuclein, insulin, lysozyme, prion 106-128 peptide and polyglutamine (KKQ40KK) and many other types of prefibrillar oligomers.

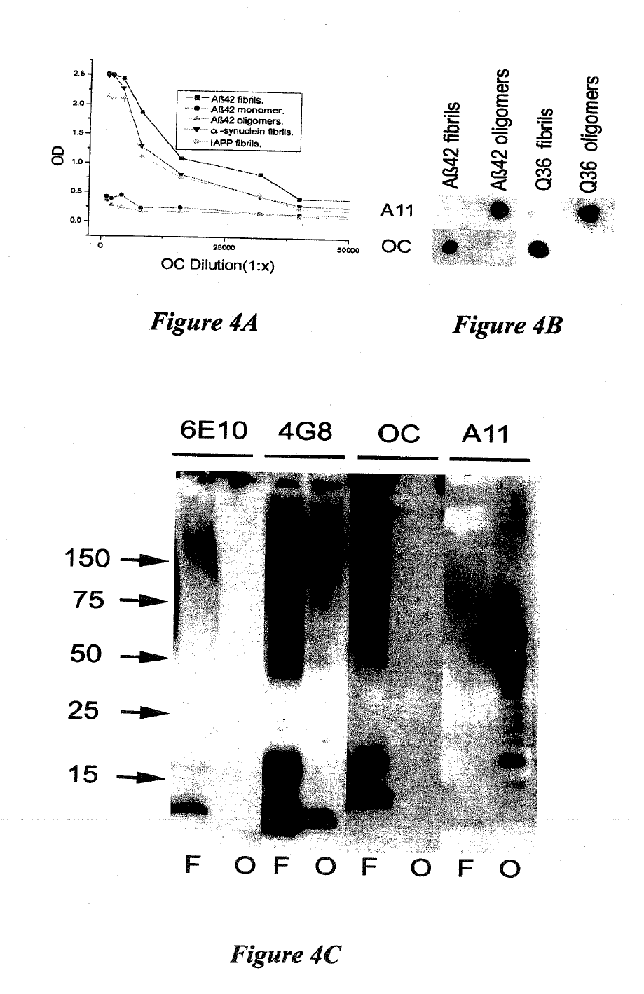

The conformation-dependent monoclonal antibodies of the present invention recognize distinct epitopes that are associated with different types of amyloids. Many of the antibodies and antisera we have produced so far recognize generic epitopes that may arise from the conformation, shape or pattern of the polypeptide aggregates and are independent of the specific amino acid sequence of the protein. These polyclonal antibodies include anti-oligomer, and two new antisera: one that recognizes an epitope found in fibrils and soluble fibril oligomers or nuclei and one associated with pore-like annular protofibrils. Amyloids of different types, including Aβ seem to share common pathways of aggregation that result in several distinct assembly states. Beginning with misfolded native states, the protein adopts an amyloidogenic conformation that facilitates its assembly along two distinct pathways as described in the Background of the Invention Section set forth above. This results in the formation of prefibrillar oligomers or the formation of an amyloid fibril lattice. We believe that these are alternative pathways, rather than prefibrillar oligomers representing an obligate intermediate in the fibril formation pathway because the formation of prefibrillar oligomers and fibrils is differentially sensitive to disruption by protein denaturants like urea and aggregation inhibitors. Prefibrillar spherical oligomers appear to be building blocks for higher order assemblies, including protofibrils and annular protofibrils because annular protofibrils can be prepared in high yield from spherical oligomers (see below). Prefibrillar oligomers and protofibrils (which appear morphologically as curvilinear strings of spherical oligomers) are unstable kinetic intermediates because they disappear as mature fibrils form (20). It is not clear whether protofibrils undergo a conformation change to become mature fibrils or whether they dissociate into monomer, which then adds on to fibril nuclei during fibril growth. Prefibrillar oligomers and protofibrils are recognized by anti-prefibrillar oligomer polyclonal antibodies (A11). Fibrils (both insoluble and 100,000×G soluble oligomeric species) are recognized by anti-fibril polyclonal antibodies (OC). Rough and smooth annular protofibrils are recognized by anti-annular protofibril polyclonal antibodies (officer). There is minimal cross-reactivity between these antibodies and the epitopes appear to be quite mutually exclusive at least at the limit of the purity of the samples used to characterize them (see FIG. 4 below).

FIGS. 4A through 4C show the specificity of anti-Fibril (OC) antibody. Specifically, FIG. 4A shows an ELISA assay of different types of fibrils and soluble Aβ monomer and prefibrillar oligomers. Wells were plated with the different samples indicated in the inset and reacted with dilutions of OC antiserum. All fibrillar samples react with OC. FIG. 4B shows a dot blot of Aβ42 fibrils and prefibrillar oligomers stained with anti-fibril (OC) or anti-oligomer (A11). OC only stains the fibrillar sample while A11 only stains the oligomeric sample. FIG. 4C shows a Western blot of fibrillar (F) and prefibrillar oligomer (O) Aβ42 samples. Prefibrillar oligomers were prepared by dilution of a stock solution of Aβ40 in urea to 45 mM in 10 mM phosphate buffer, pH 7.4 and incubation for 4 days. Both fibrillar and prefibrillar oligomer samples contain bands that react with 4G8 ranging from monomer up to the size of material that accumulates at the top of the gel. OC only stains the bands from fibrillar samples of approximately dimer and above. A11 only stains the prefibrillar oligomer samples. The sizes of the OC and A11 positive oligomers broadly overlap, although they are immunologically distinct. 6E10 does not stain prefibrillar Aβ oligomer samples formed at pH 7.4 as previously reported.

The variety of different assembly states of Aβ along with the implication that aggregation is important for the pathogenesis of AD presents an opportunity to understand their relative contribution to amyloid deposition and the mechanistic details underlying their pathogenicity. However, it also presents a challenge of how one can determine the conformational status of misfolded proteins in vivo or under physiological conditions in complex mixtures of proteins. For a long time, the conformational status has been judged by the solubility, or the size of the oligomer, according to sedimentation, size exclusion chromatography and gel electrophoresis. Although this has been useful, size measurements may lack the desired resolution and may not reflect the aggregation state under more physiological circumstances. These biochemical and biophysical measures are also more labor intensive and may not provide the required sensitivity. Conformation dependent antibodies have the potential of providing more detailed and sensitive information about the conformation of misfolded proteins.

### Oligomer Specific Antibody (A11):

We developed a conformation-dependent rabbit polyclonal antibody (A11) that specifically recognizes soluble Aβ amyloid oligomers or prefibrillar aggregates, but not natively folded APP, low molecular weight monomer and dimer, nor mature amyloid fibrils, as was recently reported. This anti-oligomer antibody specifically recognizes soluble oligomers in the same conformation-dependent fashion from many different amyloids, including lysozyme, IAPP, synuclein, prion 106-126, polyQ and insulin. This indicates that the oligomeric aggregates display a common epitope that is distinct from that displayed by mature fibrils. Because the antibody recognition is independent of the amino acid sequence, the epitope is likely to be a common peptide backbone structure, such as the edge of a β sheet or a turn motif that is either absent or structurally distinct in the fibril structure. The availability of antibodies that specifically recognize the generic oligomeric or prefibrillar assembly states of amyloids provides the opportunity to examine early aggregation events and for distinguishing the specific misfolded conformation from a vast excess of native and other folded structures in complex mixtures or tissues. Anti-oligomer antibody stains deposits of oligomers in AD brain sections that are reduced or absent from age-matched control brain and in brain tissue from transgenic mouse models of AD. The oligomeric deposits are distinct from the thioflavin-S positive fibrillar amyloid deposits and where they are found in the vicinity of diffuse amyloid deposits, their localizations remain distinct and non overlapping. Even when A11 immunoreactivity is found in the vicinity of a plaque, their distributions are distinct, with A11 being found in the periphery as if oligomers are either being recruited to a growing plaque or they are dissociating from it. At the ultrastructural level, A11 immunoreactivity has been localized in cell processes and axon terminals in human AD and Tg2576 mouse brain, suggesting that A11 positive prefibrillar oligomers may be related to neuronal dysfunction. The anti-oligomer antibody provides a facile means of assessing the significance of oligomers in pathogenesis, identifying other diseases in which oligomers are implicated, the role of oligomers in pathogenesis and potentially targeting oligomers in the development of therapeutics for amyloid-related degenerative diseases.

### Anti-Fibril Antibody (OC):

Antibodies that are specific for a generic epitope that is associated with amyloid fibrils have also been reported. We have also produced such a conformation-dependent antibody that specifically recognizes many different types of fibrils, by immunizing rabbits with homogeneous fibril preparations as described herein. This polyclonal antibody recognizes fibrils, but not prefibrillar oligomers, monomer or natively folded precursor proteins such as APP. The epitopes displayed by mature fibrils and soluble oligomers appears to be distinct and mutually exclusive as ELISA assays, dot blots and western blots of fibrillar and prefibrillar oligomers stain only with the fibril and prefibrillar oligomer antibody respectively (FIG. 2A, B, C). The size distributions of soluble Aβ species staining with these antibodies broadly overlap, indicating that size is not a good indicator of their immunologically defined conformation. This suggests that there are at least two fundamental and generic types of soluble oligomers, “prefibrillar” oligomers that stain with anti-oligomer (A11) and “fibrillar” oligomers or fibril nuclei that stain with anti-fibril (OC) and may represent small pieces of fibrils or fibril nuclei. Staining of AD brain tissue with this antibody reveals that all types of amyloid deposits stain intensely, including thioflavin negative diffuse plaques that are commonly referred to as “non-fibrillar” because of their tinctorial properties. The staining is completely blocked by preincubation of the antiserum with excess Aβ fibrils. This indicates that even though diffuse amyloid deposits are thioflavin negative, they contain the same misfolded epitope as other types of fibrillar plaques. The anti-fibril antibody also stains amylin amyloid deposits in transgenic mouse models of type II diabetes and light chain amyloid in systemic amyloidosis.

### Anti-Annular Protofibril Antibody (Officer):

Annular protofibrils are ring-like structures that resemble pores and have been described as occurring occasionally or individually in solutions containing predominantly spherical prefibrillar amyloid oligomers. Based on their morphological resemblance, it has been proposed that they represent membrane pores that can account for the membrane permeabilizing activity associated with amyloid aggregates.

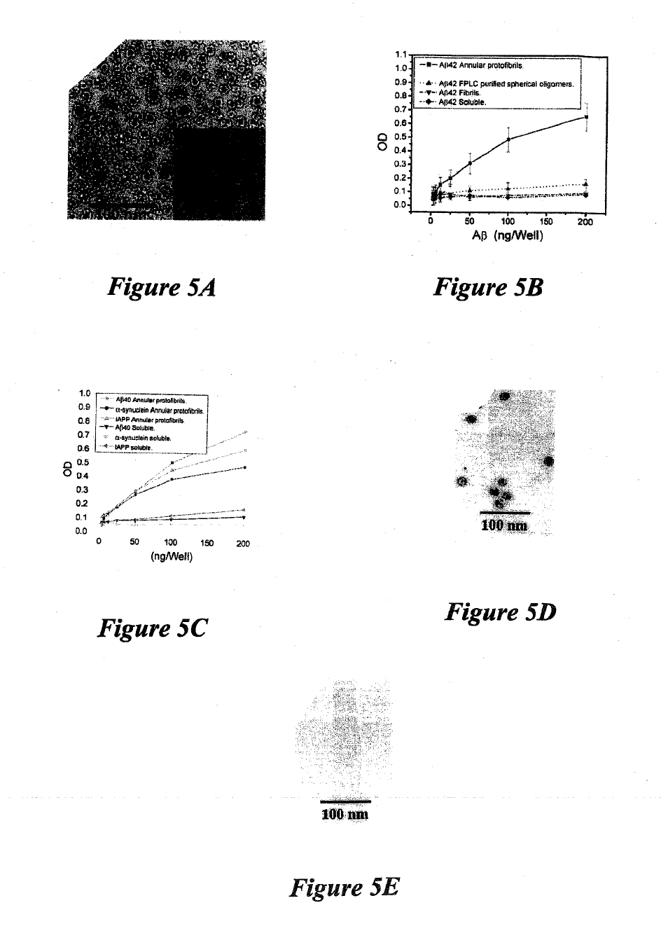

FIGS. 5A through 5E relate to these annular protofibrils and anti annular protofibrils antibody. FIG. 5A shows negative stained electron micrograph of relatively homogeneous populations of Aβ42 annular protofibrils prepared from solutions of prefibrillar oligomers by treatment with 5% hexane in water. Inset: Incubation of the sample for 2 weeks results in a loss of the beaded or rough morphology and the appearance of a smooth morphology. The same method is used for preparing annular protofibrils from alpha synuclein and IAPP. FIG. 5B shows the specificity of anti-annular protofibrils antisera. Homogeneous samples of Aβ42 annular protofibrils, spherical oligomers, fibrils and monomer (soluble) were plated in the wells of an ELISA plate and reacted with anti-annular protofibrils antibody. FIG. 5C shows that the anti-annular protofibrils antibody reacts efficiently with annular protofibrils from alpha synuclein and IAPP (amylin). FIG. 5D shows immunoprecipitation of soluble AD brain extract with anti-oligomer antibody. FIG. 5E shows immunoprecipitation of non-demented control brain extract with anti-annular protofibril antibody. 500 ul of extract with immunoprecipitated with 10 ug of anti-annular protofibrils antibody covalently attached to dynabeads. After washing the beads 3 times, the beads were eluted with 0.2 M glycine, pH 2.2 and the eluate applied to an EM grid and negatively stained. Counting of larger areas indicates a density of 60 pores/um² for the AD samples and a density of <1/um² for non-demented control brain (P<0.001).

Applicants have developed methods for preparing annular protofibrils in high yield and purity (FIG. 5A above). Homogeneous samples of annular protofibrils were used as an immunogen to raise polyclonal antisera in rabbits. The resulting antibody is remarkably selective for annular protofibrils (FIG. 5B). The shorthand laboratory jargon for this anti annular protofibril antibody is “officer” (officers like donuts). In ELISA assays using pure preparations of Aβ monomer, fibrils, prefibrillar oligomers and annular protofibrils, officer antibody is selective for annular protofibrils. There is very little reactivity against soluble monomers and fibrils, although there may be some reactivity against prefibrillar oligomers. This residual reactivity may be due to the spontaneous occurrence of annular protofibrils in oligomer preparations as previously reported. Like the anti-oligomer antibody, A11, the anti-annular protofibrils antibody specifically recognizes annular protofibrils formed from several different types of amyloidogenic proteins (FIG. 5C). We immunoprecipitated lysates of human AD and age matched control brain with officer antibody and eluted the bound fraction at low pH. Electron microscopy of the eluted fraction reveals numerous naturally occurring ring-like annular structures that have the exact same morphology and size as the aged samples prepared in vitro (compare FIGS. 5D and 5A inset). Very few if any ring-like structures were immunoprecipitated from age matched control brains (FIG. 5E).

### Monoclonal Antibodies Specific for Aβ Oligomers and Fibrils:

It is desirable to produce a number of distinct monoclonal antibodies that recognize conformation specific epitopes on amyloid aggregates. Even though the polyclonal immune response to different assembly states of amyloid is remarkably specific, monoclonal antibodies offer unique advantages in terms of defining fine structural variation in amyloid aggregates and for determining the structures of these aggregates and their pathological significance. We initially focused on antibodies against prefibrillar amyloid epitopes, by vaccinating mice with the colloidal gold coupled AF that we used to make A11. We tried to produce mouse monoclonal antibodies under contracts with two different vendors using 4 different strains of mice. Even though we obtained good titers of the expected conformation dependent IgGs in the polyclonal mouse serum, we were only able to clone IgM secreting hybridomas from the fusion products of mouse splenocytes. We tried different routes of vaccination, vaccination for 6 months and different sources of lymphocytes (peripheral and lymph node) but were never able to clone anything but IgMs. The reasons for the failure to clone IgG secreting clones from mouse hybridomas remain unclear. While these IgMs may have some utility, they are not as desirable for many applications (such as x-ray crystallography), so we contracted to make monoclonal IgGs in rabbits by contracting with Epitomics, Inc. Although we used the same antigens and screening that we used for mouse monoclonals, we obtained many more positive independent clones, many of which are IgGs. Many of these clones appear to be phenotypically identical and fall in to one of 5 or 6 distinct classes that we have identified so far.

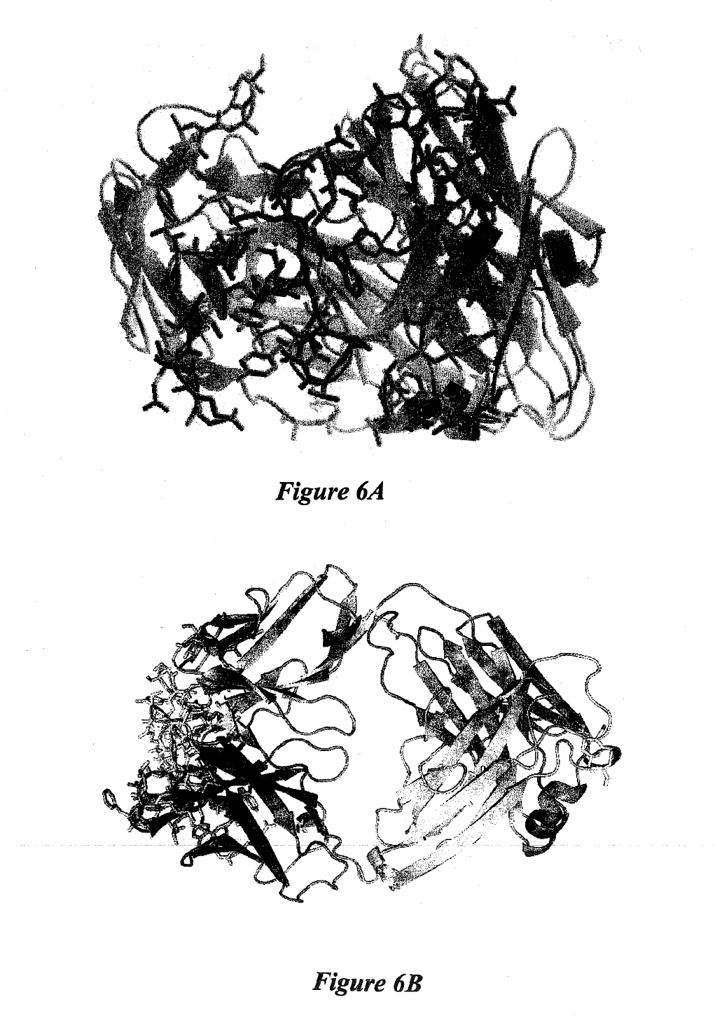

As explained in reference to FIG. 1 above, A11 stains all types of prefibrillar oligomers as previously reported, however, the rabbit monoclonal antibodies display a more restricted specificity than A11 and have distinct preferences for different types of oligomers. For example, M204 recognizes all types of oligomers, but it reacts only weakly with light chain oligomers. Structural details of M204 are shown FIGS. 6A and 6B. M201 only recognizes Aβ oligomers. M118, M205 and M206 have intermediate degrees of specificity and display distinct preferences. M121 is specific for Aβ fibrils and does not recognize Aβ monomer or prefibrillar oligomers. M201, M206 and M121 are IgMs, while the other monoclonals are IgG

The specificity of the monoclonal antibodies is of interest for several reasons. Firstly, all of the monoclonals we obtained in response to vaccination with the A11 Aβ C-terminal thioester colloidal gold antigen are conformation specific even though we selected all clones that reacted with Aβ monomer, oligomers and fibrils. None of the clones recognize monomer like 6E10. This indicates that the immune response to the solid phase antigen is highly conformation specific. None of the antibodies recognize both pure fibril and pure oligomer samples, indicating that the distribution of these epitopes is mutually exclusive. Secondly, many of the antibodies (M118, M204, M205, and M206) recognize “generic epitopes” that are distributed on prefibrillar oligomers produced from other protein and peptide sequences. However, within this class of antibodies that recognize “generic” prefibrillar oligomer epitopes there is considerable variation in the types of oligomers that the antibody recognizes. All of the generic monoclonals recognize Aβ oligomers because they were used as the primary screen, but each antibody has a specificity more restricted than the A11 polyclonal immune response. For example, M204 strongly recognizes most types of prefibrillar oligomers, but it is distinctly less reactive with immunoglobulin light chain oligomers. M205 reacts strongly with alpha synuclein and light chain oligomers, but does not react well with prion 106-126, polyQ and calcitonin prefibrillar oligomers. M118 prefers light chain and polyQ oligomers, but not synuclein, prion or calcitonin oligomers. These results indicate that there are multiple distinct epitopes associated with prefibrillar oligomers that are widely distributed within this class and that monoclonal antibodies can recognize these unique epitopes. Thirdly, some monoclonals are both conformation dependent and sequence specific. M201 recognizes only Aβ oligomers, while M121 only recognizes Aβ fibrils. M118, M204 and M205 are IgG, while the other antibodies are IgM.

### Monoclonal Antibodies Distinguish Different Subtypes of A11 Positive Prefibrillar Aβ Oligomers

When Applicants examined a large number of A11 positive, Aβ prefibrillar oligomer preparations with the monoclonal IgG antibodies, we observed that some preparations of A11 positive oligomers do not react with the monoclonal antibodies, indicating that there are immunologically distinct subclasses of Aβ prefibrillar oligomers (FIGS. 2A and 2B described above). M118 stains prefibrillar oligomers prepared at pH 2.5 and does not stain oligomers prepared at pH 7.4 (FIG. 2A). Similarly, M204 and M205 stain different preparations of Aβ42 oligomers even though they are all A11 positive. These results indicate that there are structural polymorphisms within the class of Aβ prefibrillar oligomers that can be distinguished immunologically. We have also prepared A11 positive Aβ oligomers that do not react with any of the monoclonal antibodies we have yet (FIG. 2B), indicating that there are antibodies in polyclonal A11 that remain to be cloned. While the epitope recognized by M118 is pH dependent, we do not yet know why the immunoreactivity of M204 and M205 vary because they react differentially with different preparations of Aβ oligomers prepared by the same methods.

These results were unexpected. Since A11 and OC both recognize “generic” epitopes that are mutually exclusive, the simplest interpretation would be that they recognize common differences in the peptide backbone that are distinct in prefibrillar oligomers and fibrils. The data with the monoclonals cannot be explained by the existence of a single common backbone epitope. Rather, the monoclonals recognize a set of epitopes that are specifically associated with prefibrillar oligomers and not fibrils and these epitopes are differentially displayed on prefibrillar oligomers from amyloids of different sequence and even on different oligomer preparations of the same type of peptide (Aβ). A plausible explanation for these results could be that the side chain “steric zippers” that run up and down the parallel, in register sheet constitute a major part of distinct generic epitopes. There could be a fairly large number of these epitopes, because one or more side chain zipper could constitute the epitope. The difference between the prefibrillar and fibrillar epitope could be due to differences in the backbone structure spacing of these zippers in prefibrillar oligomers and fibrils. Other potential explanations could include the existence of alpha extended strands specifically in prefibrillar oligomers or differences in the twist of the sheet between fibrils and prefibrillar oligomers.

### Electrophoretic Variability of Prefibrillar Oligomers.

Prefibrillar oligomers display a range of apparent molecular weights and electrophoretic patterns, depending on the sample preparation and on the type of gel system used. Samples prepared by dilution from a stock solution of urea into 10 mM phosphate to a final concentration 45 mM Aβ40 run as broad band centered on an apparent Mw of approximately 56 kDa, with a number of distinct lower MW bands extending as a “ladder” to approximately 16 kDa (FIG. 2C). Samples prepared by dilution of HFIP stock solution in distilled water at pH 2.5 also run as a broad band centered at approximately 56 kDa, but lack the distinct ladder of lower MW bands (FIG. 2C). Prefibrillar oligomers prepared by dilution of NaOH stock solutions of Aβ into 10 mM phosphate buffer at pH 7.4 run as a distinct ladder with a major band at an apparent MW of 14 kDa (trimer) all the way up to approximately 100 kDa. The interpretation of the exact molecular weights should be viewed with caution as there is some evidence that the electrophoretic mobility of the oligomers may not reflect their true mass. You would expect that the ladder pattern may represent oligomers that differ by a single polypeptide, but if this is true, then their electrophoretic mobility does not accurately reflect their molecular weight. There are approximately 10 discrete bands from 25 kDa to 50 kDa, indicating an apparent mass step size of 2.5 kDa, while the mass of Aβ40 is approximately 4.3 kDa. The fact that different size distributions of oligomers may be obtained by preparing oligomers under different conditions should allow us to test the role of distinct size distributions on cellular toxicity in aim 4. The finding that different size oligomers are conformationally distinct as indicated by the results with M204 staining in FIG. 5B, suggests that different size oligomers may also be conformationally distinct. No immunoreactive bands were observed on these preparations with M118 and M205, even though the samples were recognized by these antibodies on dot blots or ELISAs in the absence of SDS (data not shown). This suggests that the epitope recognized by these antibodies may be sensitive to SDS.

Additionally, immunocytochemical staining of PSAPP Tg mouse brain tissue with prefibrillar oligomer specific monoclonal antibodies indicates that anti-prefibrillar oligomer monoclonals do not stain plaques, but rather stain intracellular accumulations and display a fine punctuate distribution in synapse rich areas. In this regard, Applicants examined the distribution of the immunoreactivity of monoclonal M118 and M204 in transgenic mouse brain and in human brain. Neither antibody stains plaques. M118 exhibits intracellular staining of CA1 neurons in PSAPP Tg mouse brain. In contrast, M204 staining is finely punctuate and enriched in synaptic regions, such as the outer molecular layer. In fact the plaques stain negatively with M204, as the staining intensity of the plaques is lower than the surrounding tissue. Similar staining is observed for M205.

The structure of monoclonal M204 Fab has been determined by x-ray crystallography and this structural information is included in FIGS. 6A and 6B. FIG. 6A (View 1) is the image of the antibody fragment with the antigen combining site facing forward toward the viewer. FIG. 6B (View 2) is an image with the antigen combining site facing toward the viewer's right. In the images, the complimentary determining regions are rendered in atomic level resolution including the side chains. The other regions of the antibody fragment outside the CDRs is rendered in ribbon format.

The present invention provides for amyloid disease therapeutics which induce a specific immune response against amyloid oligomeric intermediates. Therapeutics of the invention include antibodies that specifically bind to oligomeric intermediates. Such antibodies can be monoclonal antibodies as described in this provisional patent application and/or in the above-incorporated copending U.S. patent application Ser. No. 10/572,001 or polyclonal antibodies as described in PCT International Application No. PCT/US2003/028829, which is also incorporated herein by reference. In one useful embodiment, the antibodies bind to a conformational epitope. The production of non-human monoclonal antibodies of the present invention (e.g., murine or rat) can be accomplished by, for example, immunizing the animal with an oligomeric intermediate mimic of the invention. Also contemplated is immunizing the animal with a purified amyloid intermediate.

Humanized forms of mouse antibodies of the invention can be generated by linking the CDR regions of non-human antibodies to human constant regions by recombinant DNA techniques. See Queen et al., Proc. Natl. Acad. Sci. USA 86, 10029-10033 (1989) and WO 90/07861 (incorporated by reference for all purposes).

Human antibodies may be obtained using phage-display methods. See, for example, Dower et al., WO 91/17271 and McCafferty et al., WO 92/01047. In these methods, libraries of phage are produced in which members display different antibodies on their outer surfaces. Phage displaying antibodies with a desired specificity are selected by affinity enrichment. Human antibodies against oligomeric intermediates may also be produced from non-human transgenic mammals having transgenes encoding at least a segment of the human immunoglobulin locus and an inactivated endogenous immunoglobulin locus. See, for example, Lonberg et al., WO93/12227 (1993); Kucherlapati, WO 91/10741 (1991) (each of which is incorporated by reference in its entirety for all purposes). Human antibodies can be selected by competitive binding experiments, or otherwise, to have the same epitope specificity as a particular mouse antibody. Such antibodies are particularly likely to share the useful functional properties of the mouse antibodies.

Human or humanized antibodies can be designed to have IgG, IgD, IgA and IgE constant region, and any isotype, including IgG1, IgG2, IgG3 and IgG4. Antibodies can be expressed as tetramers containing two light and two heavy chains, as separate heavy chains, light chains, as Fab, Fab′ F(ab′)₂ and Fv, or as single chain antibodies in which heavy and light chain variable domains are linked through a spacer.

In certain instances it may be desirable to combine one or more monoclonal antibodies of the present invention with a suitable carrier. Suitable carriers include serum albumins, keyhole limpet hemocyanin, immunoglobulin molecules, thyroglobulin, ovalbumin, tetanus toxoid, or a toxoid from other pathogenic bacteria, such as diphtheria, E. coli, cholera, or H. pylori, or an attenuated toxin derivative. Other carriers which may act as adjuvants for stimulating or enhancing an immune response include cytokines such as IL-1, IL-1 β and, peptides, IL-2, INF, IL-10, GM-CSF, and chemokines, such as M1P1 and and RANTES.

Human or animal subjects or patients amenable to treatment with monoclonal antibodies of the present invention include individuals at risk of amyloid disease but not showing symptoms, as well as those who already show symptoms or other evidence of amyloid disease. In the case of certain amyloid diseases including AD, virtually anyone is at risk of suffering from the disease.

Therefore, monoclonal antibodies of the present invention could be administered prophylactically, for example, as a vaccine, to the general population without any assessment of the risk of the subject patient. The present methods are especially useful for individuals who do have a known genetic risk of an amyloid disease, for example, AD. Such individuals may include those having relatives who have experienced an amyloid disease, and those whose risk is determined by analysis of genetic or biochemical markers or who exhibit symptoms or prodromes indicative of the potential for development of, or the actual presence of, such diseases. For example, genetic markers of risk toward AD include mutations in the APP gene, particularly mutations at position 717 and positions 670 and 671 referred to as the Hardy and Swedish mutations respectively (see Hardy, TINS, supra). Other markers of risk for AD are mutations in the presenilin genes, PS1 and PS2, and ApoE4, family history of AD, hypercholesterolemia or atherosclerosis.

Symptoms of amyloid disease are apparent to a physician of ordinary skill. For example, individuals presently suffering from Alzheimer's disease can be recognized from characteristic dementia, as well as the presence of risk factors described above. In addition, a number of diagnostic tests are available for identifying individuals who have amyloid diseases. For example, in the case of AD these include measurement of CSF tau and Aβ42 levels. Elevated tau and decreased Aβ42 levels signify the presence of AD.

In asymptomatic patients, treatment can begin at any age, for example, at the age of 10, 20, 30, 40, 50, 60 or 70. Treatment may entail one or more doses, for example, multiple dosages over a period of time. Treatment can be monitored by assaying antibody, or activated T-cell or B-cell responses to the therapeutic (for example, oligomeric intermediate mimic) or assaying the levels of prefibrillar aggregate present, each over time. In one embodiment, treatment by administering a single therapeutic of the invention, such as a preparation containing a single monoclonal antibody of the invention, may serve as a treatment for or preventive measure against more than one amyloid disease, for example all amyloid diseases.

In prophylactic applications, compositions of the invention or medians are administered to a patient susceptible to, or otherwise at risk of, a particular disease in an amount sufficient to eliminate or reduce the risk or delay the outset of the disease. In therapeutic applications, compositions or medians are administered to a patient suspected of, or already suffering from such a disease in an amount sufficient to cure, or at least partially arrest, the symptoms of the disease and its complications. An amount adequate to accomplish this is defined as a therapeutically- or pharmaceutically-effective dose. In both prophylactic and therapeutic regimes, therapeutics are usually administered in several dosages until a sufficient immune response has been achieved. Typically, the immune response is monitored and repeated dosages are given if the immune response starts to fade.

Effective doses of the compositions of the present invention, for the treatment of the above described conditions vary depending upon many different factors, including means of administration, target site, physiological state of the patient, whether the patient is human or animal, other medications administered, and whether treatment is prophylactic or therapeutic. Usually, the patient is a human, but in some diseases, such as mad cow disease, the patient can be a nonhuman mammal, such as a bovine or in the case of Alzheimer's disease, the patient may be a dog. Treatment dosages need to be titrated to optimize safety and efficacy. For passive immunization with an antibody, the dosage ranges from about 0.0001 mg/kg of body weight to about 100 mg/kg of body weight, and more usually about 0.01 mg/kg of body weight to about 5 mg/kg of body weight of the host. The amount of monoclonal antibody to be administered may depend on whether any adjuvant is also administered, with higher dosages being required in the absence of adjuvant. For example, 0.1 to 100 cc of a solution containing approximately 1% by weight of the desired monoclonal antibody(ies) my be injected subcutaneously, thereby delivering a dose of 1 mg to 1 g of the monoclonal antibody(ies) per injection. The timing of injections can vary significantly from once a day, to once a year, to once a decade. One typical regimen consists of an immunization followed by booster injections at 6 weekly intervals. Another regimen consists of an immunization followed by booster injections 1, 2 and 12 months later. Another regimen entails an injection every two months for life. Alternatively, booster injections can be on an irregular basis as indicated by monitoring of immune response.

Therapeutics for inducing an immune response can be administered by any suitable route of administration, for example, parenteral, topical, intravenous, oral, subcutaneous, intraperitoneal, intranasal or intramuscular. The most typical route of administration is subcutaneous although others can be equally effective. The next most common is intramuscular injection. This type of injection is most typically performed in the arm or leg muscles. Intravenous injections as well as intraperitoneal injections, intraarterial, intracranial, or intradermal injections may also be effective in generating an immune response. In some methods, therapeutics are injected directly into a particular tissue where deposits have accumulated or may accumulate.

Monoclonal antibodies of the invention can optionally be administered in combination with other agents that are at least partly effective in treatment of amyloidogenic disease. In the case of Alzheimer's and Down's syndrome, in which amyloid deposits occur in the brain, therapeutics of the invention can also be administered in conjunction with other agents that increase passage of the compositions of the invention across the blood-brain barrier. For example, as described in detail herebelow, anti-inflammatory dosages of colloidal gold or gold salts may be administered concomitantly (e.g., before, concurrently with or after) the monoclonal antibody to deter the brain inflammation associated with AD and other amyloid diseases.

Monoclonal antibodies of the invention may sometimes be administered in combination with an adjuvant. A variety of adjuvants can be used in combination with an monoclonal antibody of the invention to elicit an immune response. Preferred adjuvants augment the intrinsic response to an monoclonal antibody without causing conformational changes in the monoclonal antibody that affect the qualitative form of the response. Preferred adjuvants include alum, 3 de-O-acylated monophosphoryl lipid A (MPL) (see Great Britain Patent No. 2220211). QS21 is a triterpene glycoside or saponin isolated from the bark of the Quillaja Saponaria Molina tree found in South America (see Kensil et al., in Vaccine Design: The subunit and Ajuvant Approach (eds. Powell & Newman, Plenum Press, NY, 1995); and U.S. Pat. No. 5,057,540). Other adjuvants are oil in water emulsions, such as squalene or peanut oil, optionally in combination with immune stimulants, such as monophosphoryl lipid A. See, for example, Stoute et al., N. Engl. J. Med. (1997) 336, 86-91. Another useful adjuvant is CpG described in Bioworld Today, Nov. 15, 1998. Alternatively, a monoclonal antibody can be coupled to an adjuvant. However, such coupling should not substantially change monoclonal antibody so as to affect the nature of the immune response thereto. Adjuvants can be administered as a component of a therapeutic composition with an active agent or can be administered separately, before, concurrently with, or after administration of the therapeutic.

A preferred class of adjuvants is aluminum salts (alum), such as aluminum hydroxide, aluminum phosphate, aluminum sulfate. Such adjuvants can be used with or without other specific immunostimulating agents such as MPL or 3-DMP, QS21, polymeric or monomeric amino acids such as polyglutamic acid or polylysine.

Another class of adjuvants is oil-in-water emulsion formulations. Such adjuvants can be used with or without other specific immunostimulating agents such as muramyl peptides (for example, N-acetylmuramyl-L-threonyl-D-isoglutamine (thr-MDP), -acetyl-normuramyl-L-alanyl-D-isoglutamine (nor-MDP), N-acetylmuramyl-L-alanyl-D-isoglutamyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include (a) MF59 (WO 90/14837), containing 5% Squalene, 0.5% Tween 80 and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalane, 0.4% Tween 80.5% pluroinic-blocked polymer L121, and thr-MDP, either microfluidized into a submicron emulsion or vortexed to generate a larger particle size emulsion, and (c) Ribi™ adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™)

Another class of preferred adjuvants is saponin adjuvants, such as Stimulons (QS21, Aquila, Worcester, Mass.) or particles generated therefrom such as ISCOMs (immunostimulating complexes) and ISCOMATRIX. Other adjuvants include Complete Freund's Adjuvant (CFA) and Incomplete Freund's Adjuvant (IFA). Other adjuvants include cytokines, such as interleukins, for example, IL-1, IL-2, and IL-12, macrophage colony stimulating factor (M-CSF), tumor necrosis factor (TNF) and/or chemokines such as CXCL10 and CCL5.

An adjuvant can be administered with an monoclonal antibody as a single composition, or can be administered before, concurrent with or after administration of the monoclonal antibody. Monoclonal antibody and adjuvant can be packaged and supplied in the same vial or can be packaged in separate vials and mixed before use. Monoclonal antibody and adjuvant are typically packaged with a label indicating the intended therapeutic application. If monoclonal antibody and adjuvant are packaged separately, the packaging typically includes instructions for mixing before use. The choice of an adjuvant and/or carrier depends on the stability of the vaccine containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Optionally, two or more different adjuvants can be used simultaneously. Preferred combinations include alum with MPL, alum with QS21, MPL with QS21, and alum, QS21 and MPL together. Also, Incomplete Freund's adjuvant can be used (Chang et al., Advanced Drug Delivery Reviews 32, 173-186 (1998)), optionally in combination with any of alum, QS21, and MPL and all combinations thereof.

Compositions of the invention are often administered as pharmaceutical compositions comprising a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonmonoclonal antibodyic stabilizers and the like. However, some reagents suitable for administration to animals, such as complete Freund's adjuvant are not typically included in compositions for human use.

Pharmaceutical compositions can also include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (e.g., adjuvants).

For parenteral administration, compositions of the invention can be administered as injectable dosages of a solution or suspension of the substance in a physiologically acceptable diluent with a pharmaceutical carrier which can be a sterile liquid such as water oils, saline, glycerol, or ethanol.

Auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Compositions may be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. See Langer, Science (1990) 249, 1527 and Hanes, Advanced Drug Delivery Reviews (1997) 28, 97-119. The compositions of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to about 10%, for example, about 1% to about 2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and may contain about 10% about 95% of active ingredient, for example, about 25% to about 70%.

Topical application can result in transdermal or intradermal delivery. Topical administration can be facilitated by co-administration of the composition with cholera toxin or detoxified derivatives or subunits thereof or other similar bacterial toxins. See Glenn et al., Nature (1998) 391,851. Co-administration can be, achieved by using the components as a mixture or as linked molecules obtained by chemical crosslinking or expression as a fusion protein.

Alternatively, transdermal delivery can be achieved using a skin path or using transferosomes. See for example, Paul et al., Eur. J. Immunol. (1995) 25, 3521-24; Cevc et al., Biochem. Biophys. Acta (1998) 1368, 201-15.

### Concomitant Administration of Gold or Other Antiinflammatory Substance

The anti-inflammatory effects of gold are well established. For example, injectable colloidal gold preparations (Myochrysine™ or Solganal™) are commercially available for the treatment of rheumatoid arthritis. A gold preparation for oral administration (Auranofin™) is also available. Inflammation of in the brain is thought to be a cause or contributing factor Alzheimer's Disease, primarily because amyloid-beta (protein) which is found in the brains of Alzheimer's patients is known to be an inflammatory protein. In view of this, others have proposed the use of non-steroidal anti-inflammatory drugs such as rofecoxib (Vioxx) and naproxen (Aleve) to slow the progression of Alzheimer's Disease.

Applicants have determined, on the basis of histopathological observations, that the subcutaneous administration of colloidal gold can reduce microglial activation in the brains of mice modeling for amyloid disease. The present invention includes the administration of colloidal gold, gold salts or other antiinflammatory agents to the subject in an amount that is therapeutically effective to decrease neural inflammation. In some cases, the gold or anti-inflammatory agent may be combined with the monoclonal antibody. In other cases, the gold or anti-inflammatory agent may be administered separately from the monoclonal antibody. Any suitable dose, dosing schedule or route of administration may be used. For example, commercially available gold preparations for treatment of rheumatoid arthritis may be administered by the same routes of administration (subcutaneous injection of Myochrysine™ or Solganal™ or oral administration of Auranofin™ and dosages/dosing schedules recommended for treatment of rheumatoid arthritis.

Although the foregoing invention has been described in detail for purposes of clarity of understanding, it will be obvious that certain modifications may be practised within the scope of the appended claims. All publications and patent documents cited herein are hereby incorporated by reference in their entirety for all purposes to the same extent as if each were so individually denoted.

Systemic Immunization with Anti-Oliqomeric Monoclonal Antibodies Reduction of Beta Amyloid Deposition and Improvement of Cognitive Function in 3×Tg-AD Mice Vaccinated in Accordance with this Invention

Accumulation of the amyloid-β (A β) peptide plays a pivotal role in the pathogenesis of Alzheimer's Disease. Accordingly, immunization against A β has offered a promising approach toward the therapeutic management of Alzheimer's Disease. In animal models of Alzheimer's Disease, both active and passive anti-A β immunotherapies improve cognitive function and clear the parenchymal accumulation of amyloid (plaques) in the brain. Previously, a discontinued phase II trial of active immunization with aggregated A β as an immunogen (AN1792) along with the QS-21 adjuvant demonstrated some clinical benefit in AD subjects who developed a robust anti-A β antibody response. The autopsy reports of 4 subjects from that trial who survived up to 20 months after their first immunization revealed focal clearance of parenchymal plaques in the cortex. In contrast, the accumulation of amyloid in cerebral blood vessels, also known as cerebral amyloid angiopathy (CAA), was more severe than observed in non-immunized patients at a similar stage of AD, with multiple cortical hemorrhages evident in one case. Recent preclinical studies illustrate that passive immunizations with antibodies that target amyloid plaques and reverse the cognitive deficit also increase CAA in transgenic mouse models of Alzheimer's Disease. In addition, CAA-associated microhemorrhages were augmented with such immunizations in transgenic Alzheimer's Disease models. These effects of anti-A β immunotherapy present a significant risk in light of the fact that CAA is associated with pathological abnormalities and cognitive impairment in the elderly population, and is prevalent in 80% of patients with Alzheimer's Disease. The mechanisms proposed for the effects of anti-A β immunotherapy on amyloid accumulation remain elusive, generally relying on systemic regimens that yield high titers of anti-A β antibodies in the peripheral circulation. High antibody titers bind to substantial amounts of A β in the bloodstream, which is believed to shift the overall A β equilibrium and create a “peripheral sink” that facilitates an efflux of A β from the brain. Further, high doses of antibody in the periphery are required because of the low-level penetration of antibody across the blood-brain barrier to effectively engage the local (i.e., central) mechanisms for clearing the cerebral amyloid. Recent studies demonstrate that a single intracerebroventricular (icy) injection of anti-A β antibodies is able to prevent the Aβ-induced impairment of synaptic plasticity in the hippocampus, and also transiently reverse the memory deficit in a transgenic Alzheimer's Disease mouse model. Therefore, to gain further insight into the actions of anti-A β antibodies, Applicants have tested the short time effect of anti-oligomeric antibodies in the aged 3×Tg-AD mouse model of Alzheimer's Disease. 3×Tg-AD mice is a widely used strain that exhibits both intracellular, extracellular Aβ deposits, Tau pathology and cognitive deficits. The results of this testing show not only improvement in cognitive function but also clearance of amyloid load.

### Materials & Methods

In this study, 32 homozygous 3×Tg-AD mice (14 weeks of age) expressing mutant human genes APPswe, PS1M146V and tauP301L were used. Also, conformational specific anti-Oligomeric antibodies (A11, 204 & 205) were used.

### Immunization:

The mice were passively immunized with one of the following antibodies: A11, 204, 205 (described in Appendix A) or rabbit IgG control Ab. Immunization was for 5 weeks. The immunization was done intraperitonialy (300 μg/immunization).

### Behavioral Studies:

A total of 32 mice were pre-tested longitudinally at hidden and cued platform Morris water maze (MWM) for 7 days. The mice were divided into four groups receiving A11, 204, 205 Abs and the other group receiving vehicle Ab (rabbit IgG). In order to investigate the effect of Abs, mice were evaluated for context object recognition after every 2 weeks. Before sacrificing, the mice were again re-evaluated for MWM test. Inhibitory avoidance test (which is related to amygdala) was also done.

### Morris Water Maze Test:

The Morris water maze tests a special memory task related to hippocampal function. The apparatus used for this test was a circular aluminum tank (1.5 m diameter) painted white and filled with water maintained at 26° C.-29° C. The maze WAS located in a room containing simple visual, extramaze cues. To reduce stress, mice were placed on the platform in both the hidden and cued versions of the task for 15 sec prior to the first training trial Mice were trained to swim to a circular clear Plexiglas platform (14 cm diameter) submerged 1.5 cm beneath the surface of the water and invisible to the mice while swimming. The platform location was selected randomly for the 6 and 10 months test, but was kept constant for each individual mouse throughout training. On each trial, the mouse was placed into the tank at one of four designated start points in a pseudorandom order. Mice were allowed 60 seconds to find the submerged platform. If a mouse failed to find the platform within 60 s, it was manually guided to the platform and allowed to remain there for 15 s. After this, each mouse was placed into a holding cage under a warming lamp for 30 before beginning the next trial. To ensure that memory differences were not due to lack of task learning, mice were given four trials a day for as many days as were required to train the 3×Tg-AD-h mice to reach the criterion (<20 s). To control for overtraining, probe trials were run for each group, both as soon as they reached group criterion and after all groups had reached criterion. We trained the 3×Tg-AD for 7 days to met the criterion >20 sec. Retention of the spatial training was assessed 1.5 hr and again 24 hr after the last training trial. Both probe trials consisted of a 60 s free swim in the pool without the platform. Mice were monitored by a camera mounted in the ceiling directly above the pool to record the 1.5 hr and 24 hr test. The parameters measured during the probe trial included (1) initial latency to cross the platform location, (2) number of platform location crosses, and (3) time spent in the quadrant opposite to the one containing the platform during training. For the 10 months and 14 months training, the location of platform was changed to avoid “savings” from previous water maze experience.

### Object Recognition Test

This task is more dependent on cortical region. Each mouse was placed in the chamber with two identical objects spaced ≅12 inches apart. The animals were allowed to explore the objects for 5 min. After a 5 min retention interval in which the animal was returned to its home cage, the mouse was placed back in the chamber with the previously exposed object and a novel object for a 3 min probe test.

Passive Avoidance test

Contextual learning and memory was evaluated using the passive inhibitory avoidance task, performed in the Gemini Avoidance System (San Diego Instruments, San Diego, Calif.). The training trial consisted of placing a mouse in the illuminated compartment of the device, and recording the time required for it to enter the dark compartment (baseline latency). Upon entering, the door between the two compartments was closed and the animal was immediately given an electric shock to the feet (0.15 mA, 1 s). During the retention trials (conducted 1.5 h and 24 h after the training trial), the mouse was again placed in the illuminated compartment and the latency to enter the dark compartment was recorded. The retention trial was interrupted if the animal took more than 180 s to cross into the dark compartment

### Tissue Collection and Immunohistochemistry

Mice were deeply anesthetized with an overdose of pentobarbital (150 mg/kg, IP), blood collected by cardiac puncture, and then animals perfused transcardially with cold phosphate-buffered saline (PBS). After dissection, brain tissue was fixed overnight with 4% paraformaldehyde in PBS, pH 7.4 at 4° C. Thereafter, fixed tissue was stored in PBS/0.02% sodium azide (NaN3) at 4° C. until use. Fixed brain tissue was sectioned (40 um) with a vibratome, and coronal sections were collected in PBS (containing 0.02% sodium azide), and stored at 4° C. prior to staining. Immunohistochemistry (IHC) was performed on freefloating brain sections. To stain for Aβ plaques, sections were immersed in 50% formic acid for 5 min. Endogenous peroxidase in tissue was blocked by treating with 3% H2O2 in PBS, 10 min at room temperature. Nonspecific background staining was blocked byl hour incubation in 2% BSA with 0.3% Triton X-100 (TX) at room temperature. Sections were then incubated with primary antibodies (6E10) overnight at 4° C., rinsed 3 times with PBS with 0.1% TX and incubated with biotinylated secondary antibody followed by ABC kit reagent (Vector, Burlingame, Calif.) for 1 hour each at room temperature. Finally, after washing three times, the sections were incubated for approximately 2-5 min with diamino-benzidine (DAB), (Vector). Sections were mounted on slides, dehydrated in a series of graded ethanol, cleared with Histoclear, and then coverslipped with DePeX (Biomedical Specialities, CA).

### Quantitative Image Analysis

The NIH image processing and analysis program (available from the National Institute of Mental Health, Bethesda, Md.) was used to analyze the area occupied by β-amyloid, Immunostaining was captured using a Sony high-resolution CCD video camera (XC-77) and NIH image 1.59b5 software. For every animal, 7-8 images from the superficial layer of the frontal parietal region in the cortex were captured with a 206 objective. For hippocampal areas, similar imaging was performed for CA1, subiculum and dentate gyrus.

### Results

### Morris Water Maze-Results

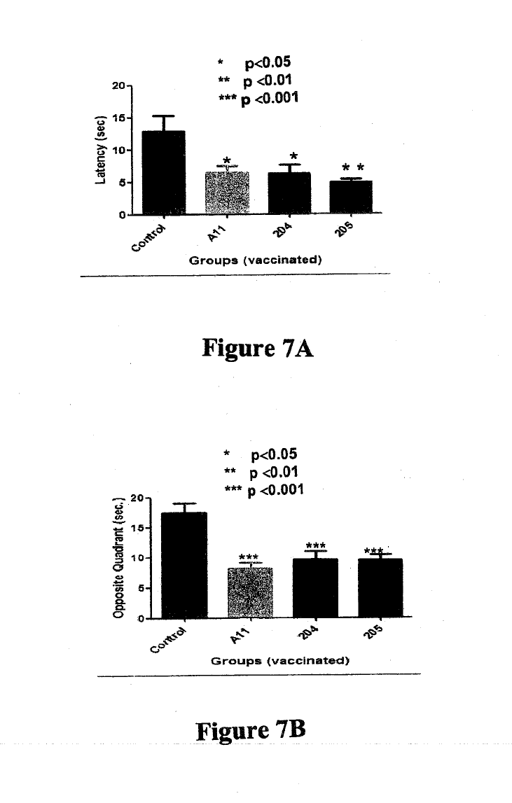

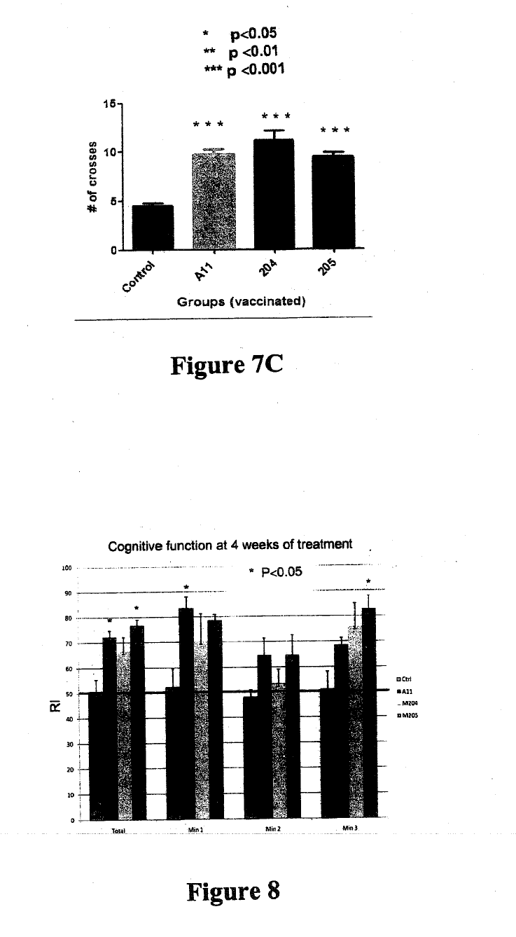

FIGS. 7A-7C are bar graphs showing the results of the Morris Water Maize test conducted 5 weeks following vaccination of mice with control (saline), A11, 204 and 205 antibodies. As shown, the mice vaccinated with A11, 204 and 205 demonstrated significantly shorter initial latency to cross the platform location (FIG. 7A), significantly less time spent during training in the quadrant opposite to the one containing the platform (FIG. 7B) and significantly greater numbers of platform location crosses (FIG. 7C). Thus, it is concluded that in this test (at the 5 week time point), A11, 204 and 205 anitbodies had significantly better retention memory than control animals.

### Object Recognition-Results

FIG. 8 is a bar graph showing Recognition Index (RI) determined by the Object Recognition test conducted 4 weeks following vaccination of mice with control (saline), A11, 204 and 205. The RI is the percentage of time spent exploring the novel object as opposed to the familiar object. Mice vaccinated with A11 and 205 antibodies had significantly higher RIs in some trials, thereby indicating potentially greater retention memory than controls.

### Passive Avoidance-Results

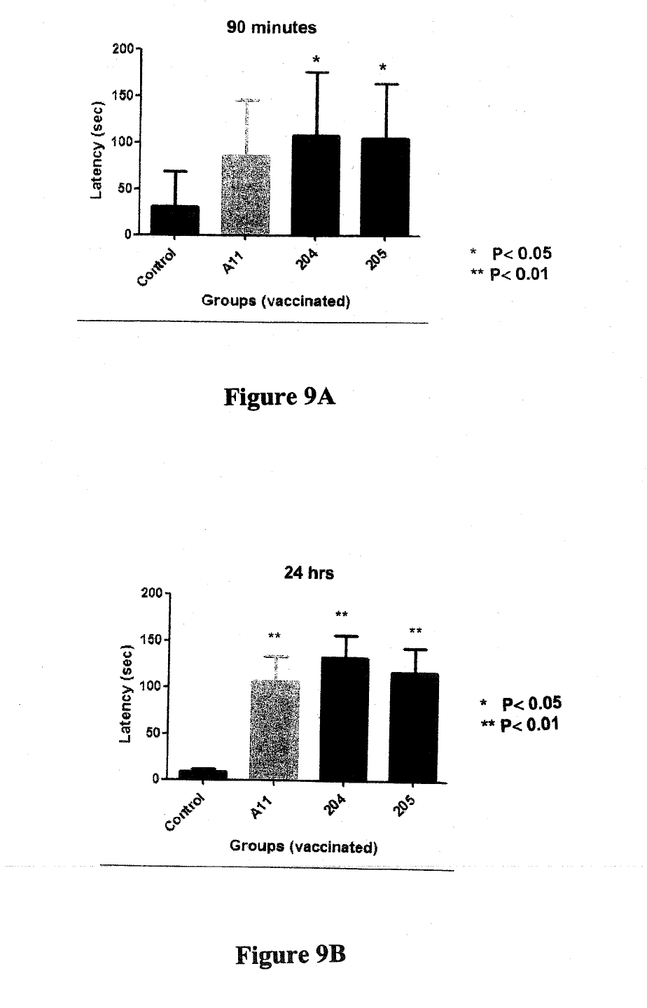

FIGS. 9A and 9B show results of the passive avoidance test. Passive avoidance memory retention (mean latency±S.E.M.) was measured as a function of each animal's ability to remember an electrical shock at 90 minutes (FIG. 9A) and 24 hours (FIG. 9B) following shock administration. As seen in FIG. 9A, at 90 minutes post shock animals vaccinated with the 204 and 205 antibodies exhibited greater latency (i.e., better passive avoidance memory) than controls. As seen in FIG. 9B, at 24 hours post shock, animals vaccinated with the A11, 204 and 205 antibodies exhibited greater latency than control animals. These data indicate that, in this test, vaccination with A11, 204 and 205 caused the animals to exhibit improved passive avoidance memory, which is believed to be mediated by neurons of the amygdala.

### Immunohistochemistry-Results

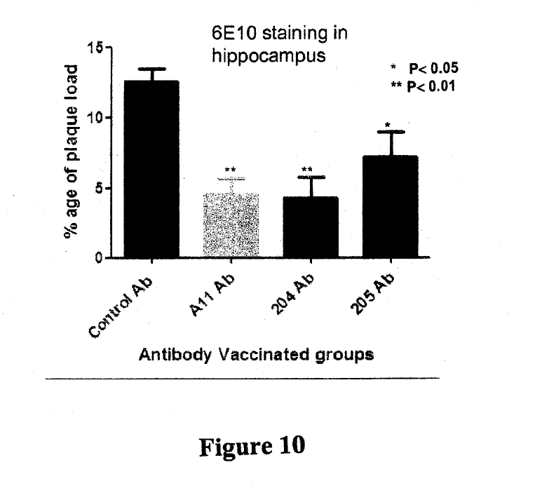

FIG. 10 is a bar graph showing Aβ plaque load determined by 6E10 antibody uptake in brain tissue of mice following vaccination with control (saline), A11, 204 and 205. In this test, animals vaccinated with A11, 204 and 205 had significantly lower Aβ plaque loads in the hippocampus than control animals. Thus, it is concluded that A11, 204 and 205 antibodies inhibited the formation of and/or caused regression of Aβ plaque in this study.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. Additionally, where a formulation or other list of components is provided or claimed, it is to be understood that such formulation or list of components may, in some embodiments, be in the absence of or to the exclusion of any or all other components that are not specifically named or indicated as being included. All reasonable additions, deletions, modifications and alterations are to be considered equivalents of the described examples and embodiments and are to be included within the scope of the following claims.

### APPENDIX A

### Antibody Production and Characterization

Two New Zealand white rabbits were vaccinated with an antigen consisting of Aβ1-40 carboxyl terminal thioester covalently bonded to colloidal gold particles via the carbosyl terminal sulfur atom. One hundred micrograms of the peptide conjugate antigen was injected with incomplete Freunds adjuvant and boosted with the same antigen at 3 week intervals for approximately 5 months. After the immune response was determined to be equivalent to the A11 antibody immune response, one of the animals was sacrificed, the spleen harvested and the splenic lymphocytes used to produce hybridomas via standard methods known in the art.

After culturing the hybridomas for a sufficient period of time, the supernatants from multiclone wells were screened for the presence of conformation dependent prefibrillar oligomer specific antibodies using Aβ prefibrillar oligomers as a primary screening agent. Aβ monomer, prefibrillar oligomers and fibrils were used as a secondary means of excluding antibodies that are not conformation dependent and interact with all Aβ conformations. Approximately 118 multiclone wells were selected as having immunoreactivity above a criterion of 0.5 AU (Table 1). These multi clones were sub-cloned and the resulting monoclones were subjected to additional screening and characterization using Aβ40 monomer, prefibrillar oligomers and fibrils.

After secondary screening, clones having a distinct preference for either prefibrillar oligomers or fibrils were selected. Representative clones selected are shown in FIG. 1 in comparison to the A11 polyclonal antibody and 6E10, a sequence dependent mouse monoclonal antibody. The conformational and sequence specificity of the clones was analyzed by dot blot. Dot blot analysis was conducted by spotting 1 ug of Aβ40 monomer, prefibrillar oligomers and fibrils and 1 ug of prefibrillar oligomers of alpha synuclein, immunoglobulin light chain, prion 106-126 peptide, KK(Q40)KK and calcitonin. All polyclonal antibody reacts with all types of prefibrillar oligomers, but not Aβ monomer or fibrils. 6E10 recognizes only samples containing Aβ. Clones 118, 201, 204, 205 and 206 are specific for prefibrillar oligomers and do not recognize monomer or fibrils. This group of clones displays distinct specificities in terms of the other types of prefibrillar oligomers recognized. Clone 121 is specific for Aβ fibrils and does not recognize prefibrillar oligomers of any type or Aβ monomer.

A number of clones appear to secrete antibodies of identical specificity. The most abundant class is similar to clone 201, which only recognize Aβ prefibrillar oligomers and not oligomers of other types FIG. 4. All of these clones are also IgMs. Clone 118 is also indistinguishable from clones 48 and 55 (data not shown).

The sequences of two of the monoclonal IgGs, 118 and 204 are shown in Table 2, below. The amino acid sequences of the variable regions is distinct, consistent with their different specificities.

### Monoclonal Antibodies Specific for Aβ Oligomers and Fibrils:

Even though the polyclonal immune response to different assembly states of amyloid is remarkably specific, monoclonal antibodies offer unique advantages in terms of defining fine structural variation in amyloid aggregates and for determining the structures of these aggregates and their pathological significance. The monoclonals obtained in response to vaccination with the A11 Aβ C-terminal thioester colloidal gold antigen are conformation specific even though we selected all clones that reacted with Aβ monomer, oligomers and fibrils. None of the clones recognize monomer like 6E10. This indicates that the immune response to the solid phase antigen is highly conformation specific. None of the antibodies recognize both pure fibril and pure oligomer samples, indicating that the distribution of these epitopes is mutually exclusive. Most of the antibodies (M118, M204, M206, M206) recognize “generic epitopes” that are distributed on prefibrillar oligomers produced from other protein and peptide sequences. However, within this class of antibodies that recognize “generic” prefibrillar oligomer epitopes there is considerable variation in the types of oligomers that the antibody recognizes. All of the generic monoclonals recognize Aβ oligomers because they were used as the primary screen, but each antibody has a specificity more restricted than the A11 polyclonal immune response. For example, M204 strongly recognizes most types of oligomers, but it is distinctly less reactive with immunoglobulin light chain oligomers. M205 reacts strongly with alpha synuclein and light chain oligomers, but does not react will with prion 106-126, polyQ and calcitonin oligomers. M118 prefers light chain and polyQ oligomers, but not synuclein, prion or calcitonin oligomers. These results indicate that there are multiple distinct epitopes associated with prefibrillar oligomers that are widely distributed within this class and that monoclonal antibodies can recognize these unique epitopes. Some monoclonals are both conformation dependent and sequence specific. M201 recognizes only Aβ oligomers, while M121 only recognizes Aβ fibrils. M118, M204 and M205 are IgG, while the other antibodies are IgM.

Monoclonal antibodies distinguish different types of A11 positive prefibrillar Aβ oligomers.

When Applicants examined a large number of A11 positive, Aβ prefibrillar oligomers with the monoclonal IgG antibodies, we observed that some preparations of A11 positive oligomers do not react with the monoclonal antibodies, indicating that there are immunologically distinct subclasses of Aβ prefibrillar oligomers (FIG. 2). M118 stains prefibrillar oligomers prepared at pH 2.5 and does not stain oligomers prepared at pH 7.4 (FIG. 2A). Similarly, M204 and M205 stain different preparations of Aβ42 oligomers even though they are all A11 positive. These results indicate that there are structural polymorphisms within the class of Aβ prefibrillar oligomers that can be distinguished immunologically. We have also prepared A11 positive Aβ oligomers that do not react with any of the monoclonal antibodies we have yet (FIG. 2B), indicating that there are antibodies in polyclonal A11 that remain to be cloned.

Anti-prefibrillar oligomer monoclonals do not stain plaques, but display a punctuate distribution in synapse rich areas.

Applicants examined the distribution of the immunoreactivity of monoclonal M118 and M204 in transgenic mouse brain and in human brain. Neither antibody stained plaques. In fact the plaques stained negatively, as they are lower than the surrounding tissue. M118 exhibited intracellular staining of CA1 neurons in both Tg mouse and human Ad brain. In contrast, M204 staining was finely punctuate and enriched in synaptic regions, such as the outer molecular layer. Similar staining was observed for M205.

TABLE 1

| | 1 | 2 | 3 | 4 | 5 | 6 | 7 | 8 | 9 | 10 | 11 | 12 |
|---|---|---|---|---|---|---|---|---|---|---|---|---|
| A | 0.24 | 0.27 | 0.10 | 0.07 | 0.05 | 0.07 | 0.09 | 0.06 | 0.14 | 0.09 | 0.14 | 1.00 |
| B | 0.04 | 0.17 | 0.13 | 0.08 | 0.05 | 0.06 | 0.07 | 0.07 | 0.10 | 0.08 | 0.21 | 0.25 |
| C | 0.09 | 0.05 | 0.07 | 0.11 | 0.05 | 0.08 | 0.06 | 0.13 | 0.07 | 0.23 | 0.11 | 0.26 |
| D | 0.12 | 0.17 | 0.05 | 0.05 | 0.06 | 0.07 | 0.06 | 0.06 | 0.08 | 0.12 | 0.15 | 0.25 |
| E | 0.14 | 0.17 | 0.07 | 0.08 | 0.07 | 0.07 | 0.10 | 0.05 | 0.08 | 0.12 | 0.16 | 0.33 |
| F | 0.18 | 0.15 | 0.07 | 0.08 | 0.04 | 0.07 | 0.09 | 0.08 | 0.15 | 0.14 | 0.14 | 0.22 |
| G | 0.18 | 0.17 | 0.07 | 0.04 | 0.04 | 0.12 | 0.09 | 0.11 | 0.10 | 0.11 | 0.22 | 0.17 |
| H | 0.22 | 0.16 | 0.05 | 0.04 | 0.06 | 0.09 | 0.13 | 0.09 | 0.19 | 0.18 | 0.21 | 0.19 |

#3/4

| | | | | | | | | | | | | |
|---|---|---|---|---|---|---|---|---|---|---|---|---|
| A | 0.04 | 0.06 | 0.07 | 0.05 | 0.06 | 0.04 | 0.05 | 0.04 | 0.04 | 0.06 | 0.11 | 0.39 |
| B | 0.04 | 0.09 | 0.08 | 0.08 | 0.07 | 0.11 | 0.07 | 0.06 | 0.07 | 0.12 | 0.07 | 0.12 |
| C | 0.04 | 0.06 | 0.05 | 0.06 | 0.07 | 0.08 | 0.06 | 0.07 | 0.07 | 0.11 | 0.07 | 0.12 |
| D | 0.03 | 0.05 | 0.06 | 0.04 | 0.05 | 0.07 | 0.06 | 0.06 | 0.06 | 0.07 | 0.09 | 0.07 |
| E | 0.10 | 0.05 | 0.07 | 0.06 | 0.06 | 0.09 | 0.07 | 0.09 | 0.07 | 0.11 | 0.10 | 0.11 |
| F | 0.06 | 0.05 | 0.04 | 0.04 | 0.05 | 0.04 | 0.04 | 0.03 | 0.06 | 0.05 | 0.13 | 0.07 |
| G | 0.03 | 0.03 | 0.05 | 0.04 | 0.14 | 0.05 | 0.04 | 0.05 | 0.06 | 0.04 | 0.04 | 0.06 |
| H | 0.04 | 0.04 | 0.06 | 0.05 | 0.08 | 0.06 | 0.05 | 0.10 | 0.08 | 0.08 | 0.07 | 0.05 |

#6/7

| | | | | | | | | | | | | |
|---|---|---|---|---|---|---|---|---|---|---|---|---|
| A | 0.03 | 0.05 | 0.02 | 0.03 | 0.02 | 0.02 | 0.02 | 0.06 | 0.03 | 0.05 | 0.05 | 0.25 |
| B | 0.03 | 0.02 | 0.02 | 0.03 | 0.04 | 0.02 | 0.02 | 0.04 | 0.03 | 0.03 | 0.04 | 0.05 |
| C | 0.03 | 0.03 | 0.02 | 0.05 | 0.02 | 0.01 | 0.02 | 0.03 | 0.03 | 0.03 | 0.03 | 0.05 |
| D | 0.04 | 0.01 | 0.01 | 0.01 | 0.02 | 0.35 | 0.01 | 0.03 | 0.01 | 0.02 | 0.03 | 0.05 |
| E | 0.02 | 0.01 | 0.00 | 0.01 | 0.01 | 0.02 | 0.01 | 0.01 | 0.01 | 0.01 | 0.02 | 0.03 |
| F | 0.02 | 0.01 | 0.00 | 0.00 | 0.03 | 0.07 | 0.01 | 0.01 | 0.01 | 0.00 | 0.02 | 0.03 |
| G | 0.01 | 0.01 | 0.01 | 0.02 | 0.00 | 0.02 | 0.01 | 0.01 | 0.01 | 0.01 | 0.01 | 0.02 |
| H | 0.02 | 0.01 | 0.01 | 0.01 | 0.01 | 0.01 | 0.01 | 0.01 | 0.02 | 0.01 | 0.01 | 0.02 |

#8/9

| | | | | | | | | | | | | |
|---|---|---|---|---|---|---|---|---|---|---|---|---|
| A | 0.02 | 0.01 | 0.02 | 0.03 | 0.03 | 0.03 | 0.02 | 0.02 | 0.01 | 0.01 | 0.01 | 0.07 |
| B | 0.00 | 0.01 | 0.01 | 0.01 | 0.00 | 0.07 | 0.01 | 0.01 | 0.01 | 0.02 | 0.03 | 0.02 |
| C | 0.02 | 0.02 | 0.01 | 0.02 | 0.02 | 0.02 | 0.02 | 0.02 | 0.03 | 0.02 | 0.02 | 0.03 |
| D | 0.02 | 0.01 | 0.01 | 0.01 | 0.02 | 0.02 | 0.02 | 0.02 | 0.03 | 0.04 | 0.03 | 0.03 |
| E | 0.03 | 0.03 | 0.04 | 0.03 | 0.03 | 0.03 | 0.05 | 0.03 | 0.03 | 0.03 | 0.03 | 0.04 |
| F | 0.03 | 0.03 | 0.03 | 0.03 | 0.03 | 0.03 | 0.02 | 0.02 | 0.03 | 0.02 | 0.04 | 0.03 |
| G | 0.03 | 0.03 | 0.03 | 0.03 | 0.04 | 0.04 | 0.03 | 0.03 | 0.03 | 0.04 | 0.02 | 0.02 |
| H | 0.01 | 0.01 | 0.02 | 0.01 | 0.01 | 0.01 | 0.02 | 0.00 | 0.00 | 0.01 | 0.01 | 0.01 |

#10/11

| | | | | | | | | | | | | |
|---|---|---|---|---|---|---|---|---|---|---|---|---|
| A | 0.24 | 0.21 | 0.09 | 0.02 | 0.02 | 0.02 | 0.02 | 0.02 | 0.02 | 0.02 | 0.02 | 0.12 |
| B | 0.01 | 0.01 | 0.01 | 0.02 | 0.02 | 0.01 | 0.03 | 0.02 | 0.02 | 0.01 | 0.02 | 0.04 |
| C | 0.01 | 0.01 | 0.01 | 0.03 | 0.02 | 0.03 | 0.02 | 0.02 | 0.01 | 0.02 | 0.03 | 0.03 |
| D | 0.02 | 0.02 | 0.01 | 0.03 | 0.01 | 0.02 | 0.01 | 0.03 | 0.02 | 0.03 | 0.02 | 0.03 |
| E | 0.02 | 0.02 | 0.02 | 0.03 | 0.02 | 0.02 | 0.02 | 0.03 | 0.02 | 0.02 | 0.02 | 0.03 |
| F | 0.02 | 0.01 | 0.01 | 0.03 | 0.02 | 0.01 | 0.01 | 0.01 | 0.01 | 0.02 | 0.02 | 0.03 |
| G | 0.02 | 0.01 | 0.02 | 0.02 | 0.01 | 0.02 | 0.01 | 0.01 | 0.02 | 0.02 | 0.02 | 0.03 |
| H | 0.01 | 0.01 | 0.02 | 0.01 | 0.01 | 0.01 | 0.02 | 0.02 | 0.03 | 0.03 | 0.03 | 0.03 |

#12/13

| | | | | | | | | | | | | |
|---|---|---|---|---|---|---|---|---|---|---|---|---|
| A | 0.08 | 0.03 | 0.02 | 0.02 | 0.02 | 0.04 | 0.06 | 0.03 | 0.03 | 0.04 | 0.02 | 0.04 |
| B | 0.02 | 0.02 | 0.02 | 0.03 | 0.02 | 0.03 | 0.02 | 0.06 | 0.04 | 0.05 | 0.03 | 0.04 |
| C | 0.03 | 0.02 | 0.02 | 0.18 | 0.01 | 0.02 | 0.01 | 0.02 | 0.08 | 0.05 | 0.03 | 0.02 |
| D | 0.08 | 0.02 | 0.02 | 0.01 | 0.01 | 0.01 | 0.02 | 0.03 | 0.02 | 0.01 | 0.02 | 0.02 |
| E | 0.03 | 0.03 | 0.02 | 0.02 | 0.02 | 0.03 | 0.02 | 0.02 | 0.02 | 0.05 | 0.01 | 0.01 |
| F | 0.05 | 0.06 | 0.02 | 0.02 | 1.46 | 0.03 | 0.02 | 0.02 | 0.03 | 0.02 | 0.02 | 0.02 |
| G | 0.05 | 0.08 | 0.04 | 0.09 | 0.02 | 0.02 | 0.02 | 0.02 | 0.04 | 0.02 | 0.02 | 0.03 |
| H | 0.18 | 0.04 | 0.03 | 0.02 | 0.02 | 0.02 | 0.01 | 0.01 | 0.01 | 0.01 | 0.01 | 0.02 |

#14/15

| | | | | | | | | | | | | |
|---|---|---|---|---|---|---|---|---|---|---|---|---|
| A | 0.19 | 0.03 | 0.01 | 0.02 | 0.02 | 0.03 | 0.02 | 0.02 | 0.02 | 0.01 | 0.01 | 0.02 |
| B | 0.03 | 0.01 | 0.01 | 0.02 | 0.01 | 0.01 | 0.02 | 0.02 | 0.02 | 0.01 | 0.02 | 0.01 |
| C | 0.02 | 0.03 | 0.04 | 0.01 | 0.02 | 0.02 | 0.01 | 0.01 | 0.01 | 0.02 | 0.02 | 0.01 |
| D | 0.02 | 0.04 | 0.04 | 0.03 | 0.02 | 0.02 | 0.01 | 0.01 | 0.01 | 0.01 | 0.01 | 0.01 |
| E | 0.05 | 0.02 | 0.02 | 0.02 | 0.02 | 0.01 | 0.01 | 0.01 | 0.02 | 0.01 | 0.01 | 0.01 |
| F | 0.04 | 0.07 | 0.03 | 0.04 | 0.02 | 0.02 | 0.02 | 0.02 | 0.01 | 0.01 | 0.01 | 0.02 |
| G | 0.09 | 0.09 | 0.04 | 0.05 | 0.02 | 0.02 | 0.03 | 0.01 | 0.01 | 0.01 | 0.01 | 0.01 |
| H | 0.28 | 0.03 | 0.01 | 0.01 | 0.02 | 0.01 | 0.01 | 0.01 | 0.01 | 0.01 | 0.00 | 0.01 |

#16/17

| | | | | | | | | | | | | |
|---|---|---|---|---|---|---|---|---|---|---|---|---|
| A | 0.20 | 0.42 | 0.35 | 0.50 | 0.44 | 0.17 | 0.10 | 0.21 | 0.22 | 0.07 | 0.08 | 0.03 |
| B | 0.47 | 0.10 | 0.09 | 0.10 | 0.12 | 0.13 | 0.05 | 0.08 | 0.07 | 0.05 | 0.05 | 0.03 |
| C | 0.90 | 0.38 | 0.74 | 0.09 | 0.08 | 0.04 | 0.03 | 0.03 | 0.02 | 0.02 | 0.04 | 0.02 |
| D | 0.30 | 0.09 | 0.18 | 0.05 | 0.18 | 0.04 | 0.03 | 0.04 | 0.01 | 0.02 | 0.02 | 0.02 |
| E | 0.11 | 0.13 | 0.12 | 0.06 | 0.07 | 0.04 | 0.06 | 0.03 | 0.01 | 0.03 | 0.06 | 0.02 |
| F | 0.11 | 0.19 | 0.11 | 0.08 | 0.05 | 0.04 | 0.07 | 0.09 | 0.02 | 0.03 | 0.02 | 0.05 |
| G | 0.12 | 0.18 | 0.06 | 0.10 | 0.03 | 0.04 | 0.04 | 0.07 | 0.10 | 0.08 | 0.03 | 0.03 |
| H | 0.56 | 0.12 | 0.09 | 0.04 | 0.03 | 0.05 | 0.07 | 0.03 | 0.03 | 0.04 | 0.09 | 0.05 |

#18/19

| | | | | | | | | | | | | |
|---|---|---|---|---|---|---|---|---|---|---|---|---|
| A | 1.54 | 1.46 | 1.29 | 0.93 | 0.03 | 1.20 | 0.04 | 0.86 | 0.07 | 0.47 | 0.48 | 0.72 |
| B | 1.16 | 0.97 | 0.93 | 0.07 | 0.97 | 0.07 | 0.07 | 0.08 | 0.03 | 0.90 | 1.30 | 0.95 |
| C | 1.20 | 0.11 | 1.12 | 1.18 | 0.16 | 0.16 | 1.01 | 0.05 | 0.04 | 0.77 | 1.08 | 0.55 |
| D | 1.61 | 0.77 | 1.09 | 1.03 | 1.10 | 0.07 | 1.21 | 1.12 | 1.08 | 1.08 | 1.36 | 0.35 |
| E | 0.17 | 0.79 | 0.75 | 0.92 | 0.87 | 0.93 | 1.03 | 0.98 | 0.06 | 0.89 | 1.11 | 1.01 |
| F | 0.14 | 0.16 | 0.07 | 0.90 | 0.85 | 1.03 | 1.09 | 1.02 | 0.04 | 0.87 | 0.97 | 0.86 |
| G | 0.89 | 0.19 | 0.15 | 0.06 | 0.92 | 1.10 | 0.91 | 0.11 | 0.71 | 0.10 | 0.14 | 0.05 |
| H | 1.70 | 1.56 | 1.25 | 1.22 | 1.24 | 1.23 | 1.22 | 1.15 | 1.27 | 1.08 | 0.19 | 0.24 |

#20/21

| | | | | | | | | | | | | |
|---|---|---|---|---|---|---|---|---|---|---|---|---|
| A | 0.22 | 0.08 | 0.08 | 1.64 | 0.11 | 0.12 | 0.14 | 0.13 | 0.11 | 0.12 | 1.84 | 2.24 |
| B | 0.05 | 0.92 | 0.08 | 0.07 | 0.15 | 0.09 | 0.14 | 0.18 | 0.16 | 0.90 | 1.81 | 1.85 |
| C | 1.62 | 0.08 | 0.15 | 0.07 | 0.45 | 0.18 | 0.16 | 0.24 | 0.14 | 0.49 | 1.76 | 0.22 |
| D | 1.55 | 0.09 | 0.11 | 0.09 | 0.39 | 0.10 | 0.13 | 0.29 | 1.75 | 0.16 | 0.20 | 0.27 |
| E | 0.06 | 0.12 | 0.10 | 0.12 | 0.29 | 0.11 | 0.13 | 1.43 | 0.16 | 0.11 | 0.15 | 0.20 |
| F | 0.10 | 0.10 | 0.11 | 0.12 | 0.12 | 0.13 | 0.99 | 1.11 | 0.14 | 0.13 | 1.57 | 1.09 |
| G | 0.15 | 0.08 | 0.94 | 1.32 | 0.17 | 0.13 | 1.20 | 1.31 | 0.09 | 0.13 | 0.11 | 0.09 |
| H | 0.12 | 1.18 | 0.11 | 1.50 | 1.69 | 0.11 | 1.77 | 1.52 | 0.10 | 1.33 | 1.36 | 0.89 |

#22/23

| | | | | | | | | | | | | |
|---|---|---|---|---|---|---|---|---|---|---|---|---|
| A | 0.06 | 0.07 | 0.09 | 0.11 | 0.13 | 0.13 | 0.13 | 0.09 | 0.13 | 0.17 | 0.13 | 0.40 |
| B | 0.07 | 0.11 | 0.08 | 0.07 | 0.12 | 0.14 | 0.12 | 0.15 | 0.25 | 0.20 | 0.20 | 0.19 |
| C | 0.08 | 0.04 | 0.10 | 0.09 | 0.08 | 0.11 | 0.10 | 0.14 | 0.14 | 0.24 | 0.25 | 0.26 |
| D | 0.06 | 0.05 | 0.16 | 0.10 | 0.16 | 0.10 | 0.11 | 0.16 | 0.21 | 0.28 | 0.40 | 0.21 |
| E | 0.09 | 0.08 | 0.07 | 0.09 | 0.11 | 0.09 | 0.08 | 0.15 | 0.13 | 0.18 | 0.28 | 0.21 |
| F | 0.08 | 0.05 | 0.15 | 0.14 | 0.15 | 0.12 | 0.16 | 0.12 | 0.12 | 0.11 | 0.12 | 0.15 |
| G | 0.08 | 0.08 | 0.06 | 0.24 | 0.14 | 0.14 | 0.12 | 0.14 | 0.15 | 0.13 | 0.12 | 0.12 |
| H | 0.12 | 0.17 | 0.20 | 0.24 | 0.45 | 0.19 | 0.29 | 0.29 | 0.39 | 0.66 | 0.40 | 0.29 |

#24/26

| | | | | | | | | | | | | |
|---|---|---|---|---|---|---|---|---|---|---|---|---|
| A | 0.18 | 0.03 | 0.04 | 0.02 | 0.04 | 0.03 | 0.04 | 0.05 | 0.08 | 0.09 | 0.11 | 0.68 |
| B | 0.05 | 0.05 | 0.04 | 0.02 | 0.02 | 0.04 | 0.04 | 0.05 | 0.08 | 0.08 | 0.11 | 0.24 |
| C | 0.03 | 0.04 | 0.03 | 0.47 | 0.03 | 0.06 | 0.05 | 0.06 | 0.09 | 0.09 | 0.13 | 0.35 |
| D | 0.03 | 0.04 | 0.04 | 0.06 | 0.06 | 0.03 | 0.05 | 0.06 | 0.08 | 0.13 | 0.12 | 0.19 |
| E | 0.04 | 0.02 | 0.02 | 0.04 | 0.04 | 0.05 | 0.04 | 0.04 | 0.10 | 0.09 | 0.10 | 0.16 |
| F | 0.06 | 0.03 | 0.03 | 0.06 | 0.05 | 0.05 | 0.07 | 0.08 | 0.06 | 0.08 | 0.11 | 0.24 |
| G | 0.08 | 0.04 | 0.04 | 0.04 | 0.06 | 0.06 | 0.05 | 0.06 | 0.10 | 0.08 | 0.10 | 0.30 |
| H | 0.05 | 0.04 | 0.08 | 0.06 | 0.10 | 0.09 | 0.11 | 0.08 | 0.09 | 0.12 | 0.08 | 0.24 |

#27/28

| | | | | | | | | | | | | |
|---|---|---|---|---|---|---|---|---|---|---|---|---|
| A | 0.02 | 0.02 | 0.01 | 0.00 | 0.00 | 0.00 | 0.01 | 0.00 | 0.01 | 0.01 | 0.01 | 0.05 |
| B | 0.00 | 0.00 | 0.01 | 0.00 | 0.01 | 0.01 | 0.01 | 0.01 | 0.01 | 0.01 | 0.01 | 0.01 |
| C | 0.00 | 0.00 | 0.01 | 0.04 | 0.00 | 0.19 | 0.00 | 0.00 | 0.01 | 0.01 | 0.01 | 0.02 |
| D | 0.00 | 0.00 | 0.00 | 0.01 | 0.01 | 0.05 | 0.01 | 0.01 | 0.01 | 0.01 | 0.01 | 0.01 |
| E | 0.01 | 0.00 | 0.00 | 0.00 | 0.00 | 0.00 | 0.01 | 0.01 | 0.00 | 0.01 | 0.01 | 0.01 |
| F | 0.01 | 0.00 | 0.00 | 0.00 | 0.00 | 0.00 | 0.00 | 0.01 | 0.01 | 0.01 | 0.01 | 0.01 |
| G | 0.01 | 0.00 | 0.00 | 0.00 | 0.00 | 0.00 | 0.00 | 0.00 | 0.00 | 0.01 | 0.01 | 0.01 |
| H | 0.01 | 0.01 | 0.00 | 0.01 | 0.03 | 0.01 | 0.01 | 0.01 | 0.01 | 0.01 | 0.05 | 0.01 |

#29/30

| | | | | | | | | | | | | |
|---|---|---|---|---|---|---|---|---|---|---|---|---|
| A | 0.09 | 0.04 | 0.05 | 0.21 | 0.08 | 0.07 | 0.10 | 0.21 | 0.09 | 0.09 | 0.09 | 0.37 |
| B | 0.06 | 0.06 | 0.03 | 0.09 | 0.09 | 0.11 | 0.29 | 0.13 | 0.09 | 0.18 | 0.12 | 0.25 |
| C | 1.01 | 0.04 | 0.04 | 0.03 | 0.03 | 0.10 | 0.09 | 0.13 | 0.07 | 0.13 | 0.15 | 0.32 |
| D | 0.03 | 0.03 | 0.05 | 0.03 | 0.07 | 0.12 | 0.07 | 0.07 | 0.08 | 0.09 | 0.07 | 0.23 |
| E | 0.05 | 0.06 | 0.06 | 0.04 | 0.06 | 0.07 | 0.07 | 0.12 | 0.12 | 0.12 | 0.04 | 0.13 |
| F | 0.06 | 0.05 | 0.07 | 0.07 | 0.13 | 0.09 | 0.07 | 0.12 | 0.04 | 0.06 | 0.05 | 0.19 |
| G | 0.05 | 0.04 | 0.11 | 0.08 | 0.05 | 0.04 | 0.05 | 0.04 | 0.08 | 0.06 | 0.05 | 0.09 |
| H | 0.11 | 0.05 | 0.07 | 0.10 | 0.08 | 0.13 | 0.09 | 0.21 | 0.23 | 0.16 | 0.11 | 0.15 |

#31/32

| | | | | | | | | | | | | |
|---|---|---|---|---|---|---|---|---|---|---|---|---|
| A | 0.09 | 0.07 | 0.08 | 0.06 | 0.10 | 0.08 | 0.09 | 0.15 | 0.37 | 0.07 | 0.10 | 0.29 |
| B | 0.07 | 0.06 | 0.10 | 0.07 | 0.03 | 0.09 | 0.10 | 0.06 | 0.10 | 0.09 | 0.11 | 0.33 |
| C | 0.06 | 0.05 | 0.08 | 0.07 | 0.50 | 0.06 | 0.07 | 0.06 | 0.08 | 0.15 | 0.06 | 0.13 |
| D | 0.06 | 0.04 | 0.08 | 0.06 | 0.09 | 0.04 | 0.06 | 0.04 | 0.03 | 0.05 | 0.12 | 0.13 |
| E | 0.06 | 0.05 | 0.04 | 0.06 | 0.04 | 0.05 | 0.06 | 0.05 | 0.07 | 0.14 | 0.10 | 0.12 |
| F | 0.17 | 0.08 | 0.06 | 0.05 | 0.11 | 0.07 | 0.05 | 0.14 | 0.04 | 0.05 | 0.05 | 0.11 |
| G | 0.09 | 0.68 | 0.13 | 0.12 | 0.08 | 0.06 | 0.10 | 0.07 | 0.08 | 0.07 | 0.06 | 0.12 |
| H | 0.15 | 0.08 | 0.09 | 0.11 | 0.13 | 0.17 | 0.12 | 0.09 | 0.11 | 0.09 | 0.07 | 0.09 |

#33/34

| | | | | | | | | | | | | |
|---|---|---|---|---|---|---|---|---|---|---|---|---|
| A | 0.03 | 0.04 | 0.02 | 0.03 | 0.04 | 0.04 | 0.05 | 0.05 | 0.12 | 0.05 | 0.12 | 0.17 |
| B | 0.06 | 0.07 | 0.04 | 0.05 | 0.10 | 0.29 | 0.10 | 0.22 | 0.05 | 0.07 | 0.14 | 0.19 |
| C | 0.03 | 0.05 | 0.02 | 0.03 | 0.04 | 0.05 | 0.05 | 0.03 | 0.04 | 0.05 | 0.08 | 0.11 |
| D | 0.03 | 0.05 | 0.03 | 0.02 | 0.03 | 0.03 | 0.03 | 0.04 | 0.03 | 0.03 | 0.08 | 0.19 |
| E | 0.02 | 0.02 | 0.01 | 0.02 | 0.13 | 0.02 | 0.10 | 0.03 | 0.01 | 0.02 | 0.06 | 0.06 |
| F | 0.04 | 0.02 | 0.01 | 0.01 | 0.02 | 0.09 | 0.05 | 0.03 | 0.03 | 0.03 | 0.06 | 0.17 |
| G | 0.02 | 0.02 | 0.04 | 0.04 | 0.07 | 0.01 | 0.04 | 0.03 | 0.04 | 0.04 | 0.07 | 0.07 |
| H | 0.15 | 0.02 | 0.04 | 0.04 | 0.06 | 0.03 | 0.05 | 0.03 | 0.06 | 0.02 | 0.08 | 0.08 |

#35/36

| | | | | | | | | | | | | |
|---|---|---|---|---|---|---|---|---|---|---|---|---|
| A | 0.16 | 0.08 | 0.14 | 0.21 | 0.18 | 0.12 | 0.12 | 0.16 | 0.20 | 0.17 | 0.30 | 1.06 |
| B | 0.16 | 0.12 | 0.08 | 0.10 | 0.09 | 0.13 | 0.14 | 0.16 | 0.28 | 0.38 | 0.31 | 0.40 |
| C | 0.13 | 0.08 | 0.10 | 0.09 | 0.07 | 0.12 | 0.12 | 0.12 | 0.17 | 0.23 | 0.35 | 0.34 |
| D | 0.09 | 0.12 | 0.09 | 0.10 | 0.19 | 0.10 | 0.09 | 0.14 | 0.20 | 0.18 | 0.29 | 0.39 |
| E | 0.10 | 0.08 | 0.09 | 0.10 | 0.11 | 0.11 | 0.11 | 2.94 | 0.17 | 0.11 | 0.12 | 0.35 |
| F | 1.05 | 0.54 | 1.03 | 0.16 | 0.07 | 0.11 | 0.11 | 0.13 | 0.15 | 0.12 | 0.37 | 0.23 |
| G | 0.15 | 0.10 | 0.15 | 0.13 | 0.09 | 0.07 | 0.09 | 0.13 | 0.15 | 0.14 | 0.12 | 0.43 |
| H | 0.12 | 0.09 | 0.11 | 0.09 | 0.13 | 0.11 | 0.08 | 0.16 | 0.10 | 0.09 | 0.23 | 0.18 |

#37/38

| | | | | | | | | | | | | |
|---|---|---|---|---|---|---|---|---|---|---|---|---|
| A | 0.31 | 0.04 | 0.61 | 0.03 | 0.04 | 0.54 | 0.11 | 0.06 | 0.13 | 0.11 | 0.42 | 1.19 |
| B | 0.03 | 0.02 | 0.03 | 0.04 | 0.04 | 0.05 | 0.38 | 0.15 | 0.12 | 0.08 | 0.61 | 0.12 |
| C | 0.03 | 0.03 | 0.04 | 0.03 | 0.06 | 0.15 | 0.10 | 0.34 | 0.17 | 0.55 | 0.21 | 0.28 |
| D | 0.04 | 0.03 | 0.04 | 0.04 | 0.03 | 0.09 | 0.11 | 0.30 | 0.09 | 0.15 | 0.20 | 0.57 |
| E | 0.03 | 0.03 | 0.02 | 0.13 | 0.17 | 0.16 | 0.09 | 0.08 | 0.13 | 0.45 | 0.16 | 0.20 |
| F | 0.04 | 0.04 | 0.03 | 0.05 | 0.12 | −0.02 | 0.05 | 0.07 | 0.36 | 0.11 | 0.05 | 0.09 |
| G | 0.05 | 0.54 | 0.36 | 0.04 | 0.51 | 0.09 | 0.16 | 0.12 | 0.07 | 0.06 | 0.55 | 0.23 |
| H | 0.03 | 0.05 | 0.06 | 0.07 | 0.05 | 0.37 | 0.41 | 0.08 | 0.13 | 0.11 | 0.12 | 0.15 |

#39/40

| | | | | | | | | | | | | |
|---|---|---|---|---|---|---|---|---|---|---|---|---|
| A | 0.77 | 0.23 | 0.26 | 0.36 | 0.15 | 0.38 | 0.23 | 0.46 | 0.26 | 0.24 | 0.19 | 0.88 |
| B | 0.16 | 0.12 | 0.19 | 0.15 | 0.18 | 0.13 | 0.18 | 0.48 | 0.21 | 0.25 | 0.21 | 0.31 |
| C | 0.13 | 0.14 | 0.18 | 0.12 | 0.13 | 0.32 | 0.15 | 0.20 | 0.39 | 0.23 | 0.49 | 0.24 |
| D | 0.33 | 0.15 | 0.26 | 0.16 | 0.18 | 0.16 | 0.18 | 0.49 | 0.69 | 0.19 | 0.21 | 0.39 |
| E | 0.15 | 0.08 | 0.21 | 0.13 | 0.17 | 0.20 | 0.18 | 0.23 | 0.45 | 0.18 | 0.21 | 0.29 |
| F | 0.11 | 0.05 | 0.08 | 0.19 | 0.20 | 0.20 | 0.25 | 0.20 | 0.21 | 0.17 | 0.26 | 0.17 |
| G | 0.15 | 0.91 | 0.32 | 0.15 | 0.29 | 0.15 | 0.25 | 0.24 | 0.17 | 0.20 | 0.28 | 0.34 |
| H | 0.13 | 0.08 | 0.09 | 0.25 | 0.14 | 0.26 | 0.21 | 0.21 | 0.18 | 0.44 | 0.38 | 0.39 |
| | CTRL | | | | | | | | | | | |

(+) = 0.5 < O.D. < 1.0
(++) = O.D. > 1.0

TABLE 2
Sequences of monoclonal antibodies
118, 121, 201, 204 and 205.

SEQ ID 10
Monoclonal Antibody #118 kappa Vl + Cl
AQAAELVMTQTPASVSAAVGGTVTINCQSSESVYNSRLSWFQQKPGQPP
KLLIYFASTLASGVSSRFSGSGSGTEFTLTISGVQCDDAATYYCAGHFS
NSVYTFGGGTEVVVTGDPVAPTVLIFPPSADLVATGTVTIVCVANKYFP
DVTVTWEVDGTTQTTGIENSKTPQNSADCTYNLSSTLTLTSTQYNSHKE
YTCKVTQGTTSVVQSFNRGDC*
SEQ ID 11
Monoclonal Antibody #118 Vh + Clh
AAQPAMAQSVEESGGRLVTPGTPLTLTCTVSGFSLSAYEVSWVRQAPGK
GLEWIGIIYANGNTVYASWAKGRFTISKTSTKVDLRIPSPTTEDTATYF
CARDIYTTTTNLWGPGTLVTVSSGQPKAPSVFPLAPCCGDTHSSTVTLG
CLVKGYLPEPVTVTWNSGTLTNGVRTFPSVRQSSGLYSLSSVVSVTSSS
QPVTCNVAHPATNTKVDKTVAPSTCSKTSC
SEQ ID 12
Monoclonal Antibody #121 kappa Vl + Cl
AQAAELVMTQTPSSVSEPVGGTVTIKCQASQSIDSYLSWYQQKPGQPPK
LLIYSASTLASGVPSRFKGSGSGTQFTLTISDLECADAATYYCQSNYWS
TSYSYAVPFGGGTEVVVKGDPVAPTVLIFPPSADLVATGTVTIVCVANK
YFPDVTVTWEVDGTTQTTGIENSKTPQNSADCTYNLSSTLTLTSTQYNS
HKEYTCKVTQGTTSVVQSFNRGDc*
SEQ ID 13
Monoclonal Antibody #121 Vh + Clh (IgM)
AAQPAMAQLVEESGGRLVTPGTPLTLTCTVSGFSLSSYAMSWVRQAPGK
GLEWIGVISSSGNTYYASWAKGRFTISKTSTTVDLKMTSLTTEDTATYF
CARGVVSGRIDTGLTLWGPGTLVTVSSVSLSSPTLYPLVSCEGALTDGN
LVAMGCLARDFLPSSITFSWSFKNNSEISSRTVRTFPVVKRGDKYMATS
QVLVPSKDVLQGTEEYLVCKVQHDNSNRDLRVSFPGEPRGLPRDGEVDS
ELPTSV
SEQ ID 14
Monoclonal Antibody #201 kappa Vl + Cll
AQAAELVMTQTPSSVSEPVGGTVTIKCQASQSIDSYLSWYQQKPGQPPK
LLIYSASTLASGVPSRFKGSGSGTQFTLTISDLECADAATYYCQSNYWS
TSYSYAVPFGGGTEVVVKGDPVAPTVLIFPPSADLVATGTVTIVCVANK
YFPDVTVTWEVDGTTQTTGIENSKTPQNSADCTYNLSSTLTLTSTQYNS
HKEYTCKVTQGTTSVVQSFNRGDC*
SEQ ID 15
Monoclonal Antibody #204 kappa Vl + Cll
AQAAELDMTQTPASVSEPVGGTVTIKCQASQSISSYLAWYQQKPGQRPR
LLIYETSTLASGVPSRFKGSGSGTEFTLTISDLECADAATYYCQSTYEN
PTYVSFGGGTEVGVKGDPVAPTVLIFPPSADLVATGTVTIVCVANKYFP
DVTVTWEVDGTTQTTGIENSKTPQNSADCTYNLSSTLTLTSTQYNSHKE
YTCKVTQGTTSVVQSFNRGDC*
SEQ ID 16
Monoclonal Antibody #204 Vh + Clh
AAQPAMAQSVKESGGRLVTPGTPLTLACTVSGFSLNTYSMFWVRQAPGK
GLQWIGIISNFGVIYYATWAKGRFTISKTSTTVDLKITSPTTEDTATYF
CVRKYGSEWGGDLWGPGTLVTVSSGQPKAPSVFPLAPCCGDTPSSTVTL
GCLVKGYLPEPVTVTWNSGTLTNGVRTFPSVRQSSGLYSLSSVVSVTSS
SQPVTCNVAHPATNTKVDKTVAPSTCSKTSC
SEQ ID 17
Monoclonal Antibody #205 kappa Vl + Cll
AQAAELVMTQTPSSVSAAVGGTVTISCQSSESVYNNNYLSWYQQKPGQP
PKRLIDSASTLDSGVPSRFKGSGSGAQFTLTISDLECDDAATYYCAGAY
VNWMRIFGGGTEVVVKGDPVAPTVLIFPPSADLVATGTVTIVCVANKYF
PDVTVTWEVDGTTQTTGIENSKTPQNSADCTYNLSSTLTLTSTQYNSHK
EYTCKVTQGTTSVVQSFNRGDC*
SEQ ID 18
Monoclonal Antibody #205 Vh + Clh
MAQSVEESGGRLVTPGTPLTLTCTASGFSLINYYMNWVRQAPGKGLEWI
GLITGWADTYYANSAKGRFTISKTSSTTVDLKITSPTTDDTATYFCVRG
GHTNIISLWGPGTLVTVSSGQPKAPSVFPLPPCCGDTPSSTVTLGCLVK
GYLPEPVTVTWNSGTLTNGVRIFPSVRQSSGLYSLSSVVSVTSSSQPVT
CNVAHPATNTKVDKTVAPSTCSKTSC

1. A composition comprising an isolated monoclonal antibody which binds to a conformational epitope of a prefibrillar aggregate which forms in a human or animal contributing to amyloid fibril formation, said monoclonal antibody being specific for a conformation-dependent epitope that is preferentially displayed by oligomeric conformations of Aβ and other amyloids.

2-14. (canceled)

15. A composition comprising a monoclonal antibody which binds to an epitope of a prefibrillar aggregate which forms in a human or animal contributing to an amyloid fibril formation wherein the amyloid fibril is substantially free of the epitope.

16-29. (canceled)

30. A method for treating a disease or condition characterized by amyloid deposits in a human or animal subject, said method comprising the step of:
A. causing a monoclonal antibody to bind to a conformational epitope of a prefibrillar aggregate which forms in a human or animal contributing to fibril formation.

31-49. (canceled)

50. A method for treating a disease or condition characterized by amyloid deposits neural tissue in a human or animal subject, said method comprising the step of:
A. causing a monoclonal antibody to bind to an epitope of a prefibrillar aggregate which forms in a human or animal contributing to an amyloid fibril formation wherein the amyloid fibril is substantially free of the epitope.

51-70. (canceled)

71. A method for making a monoclonal antibody, said method comprising the step of:
A. obtaining a conformational epitope of a prefibrillar aggregate which forms in a human or animal contributing to amyloid fibril formation.

72. (canceled)

73. A method for making a monoclonal antibody, said method comprising the step of:
A. administering to a human or animal a composition comprising an epitope of a prefibrillar aggregate which forms in a human or animal contributing to an amyloid fibril formation wherein the amyloid fibril is substantially free of the epitope.

74. (canceled)

75. A method for diagnosing a disease or condition in a human or animal subject, said disease or condition being characterized by the formation of amyloid deposits in neural tissue, said method comprising the step of:
A. combining tissue or fluid from the human or animal subject and a composition comprising or consisting of a monoclonal antibody, said monoclonal antibody being one that binds to a conformational epitope of a prefibrillar aggregate that contributes to amyloid fibril formation.

76-78. (canceled)

79. A method for diagnosing a disease or condition in a human or animal subject, said disease or condition being characterized by the formation of amyloid deposits in neural tissue, said method comprising the step of:
A. combining tissue or fluid from a human or animal subject and a composition comprising a monoclonal antibody which binds to an epitope of a prefibrillar aggregate which forms in a human or animal contributing to an amyloid fibril formation wherein the amyloid fibril is substantially free of the epitope.

80-82. (canceled)

83. A diagnostic kit useful for detecting a disease or condition characterized by amyloid deposits in the central nervous system of a human or animal subject, said kit comprising:
a composition that consists of or comprises a monoclonal antibody which binds to a conformational epitope of a prefibrillar aggregate which forms in the human or animal subject and contributes to amyloid fibril formation.

84-86. (canceled)

87. A diagnostic kit useful for detecting a disease or condition characterized by amyloid deposits in the central nervous system of a human or animal subject, said kit comprising:
an isolated composition comprising a monoclonal antibody which binds to an epitope of a prefibrillar aggregate which contributes to amyloid fibril formation.

88-90. (canceled)

91. A method for treating or preventing Alzheimer's Disease and/or another amyloid disease which causes brain inflammation in a human or animal subject, said method comprising the steps of:
A) administering to the subject a therapeutically effective amount of a monoclonal antibody composition according to claim 1; and
B) administering to the subject an antiinflammatory agent in an amount that is effective to deter brain inflammation.

92-97. (canceled)
